(19) United States
(12) Patent Application Publication
(10) Pub. No.: US 2026/0209839 A1
(43) Pub. Date: Jul. 23, 2026

(54) Methods of Sequencing Polypeptides and Related Compositions

(71) Applicant: The Board of Trustees of the Leland Stanford Junior University, Stanford, CA (US)

(72) Inventors: Hyongsok Soh, Santa Barbara, CA (US); Liwei Zheng, Redwood City, CA (US); Yujia Sun, Redwood City, CA (US); Brandon Wilson, Redwood City, CA (US); Joseph Buonomo, Redwood City, CA (US)

(21) Appl. No.: 19/157,870

(22) PCT Filed: Feb. 23, 2024
(86) PCT No.: PCT/US2024/017167

§ 371 Date: Aug. 19, 2025

Related U.S. Application Data
(60) Provisional application No. 63/448,131, filed on Feb. 24, 2023.

(51) Int. Cl. C12Q 1/6869; C12Q 1/682

(52) U.S. Cl. CPC C12Q 1/6869; C12Q 1/682

(57) ABSTRACT

Provided are methods of sequencing a polypeptide. In certain embodiments, the methods comprise labeling the N-terminal amino acid of a polypeptide with a nucleic acid label comprising a unique molecular identifier and cycle number barcode; degrading the N-terminal amino acid from the polypeptide; annealing a primer to the nucleic acid label of the degraded amino acid, where the primer comprises a barcode corresponding to the identity of the degraded N-terminal amino acid; and extending the primer annealed to the nucleic acid label to produce an extension product comprising the unique molecular identifier, the cycle number barcode, and the barcode corresponding to the identity of the degraded N-terminal amino acid. The preceding steps are performed successively to produce a plurality of such extension products which are then sequenced, enabling determination of the amino acid sequence of the polypeptide. Composition and kits that find use in practicing the methods are also provided.

### CROSS-REFERENCE TO RELATED APPLICATIONS

This application claims the benefit of U.S. Provisional Patent Application No. 63/448,131, filed Feb. 24, 2023, which application is incorporated herein by reference in its entirety.

### INTRODUCTION

Over the past 10 years, advancements in DNA sequencing have enabled mRNA sequencing at the single-cell level, and this has revolutionized our understanding of heterogeneity of biological systems. The impact of these insights on healthcare is significant—ranging from our fundamental understanding of early organ development to identification of rare treatment-resistant cell populations inside complex tumors. Historically, researchers have assumed that mRNA expression and protein expression levels are directly correlated. However, across-gene correlation analyses have demonstrated that mRNA levels only explain around 40% of the variability in protein levels. This is because protein levels are affected by many factors such as translation rate, translation modulation, and protein degradation. Furthermore, proteins often undergo post-translational modifications (PTMs) after synthesis such as phosphorylation, glycosylation, methylation and many others. It is well-known that PTMs can have a dramatic impact on protein activity, localization, and interactions with other biomolecules. Critically, PTMs are regulated by enzymatic processes, which are not directly encoded by genes and thus they cannot be predicted from the transcriptome. Thus, there is an urgent unmet need to go beyond mRNA sequencing and to directly sequence proteins (and their PTMs) at the single-cell level.

Since the 1970's protein sequencing has been performed through mass spectrometry (MS) and the sensitivities of MS instruments have dramatically increased over the past five decades. For instance, current advanced MS instruments can detect ~1000 different proteins in 0.8 ng of cell lysate. Although this is impressive, they only account for a small fraction of ~20,000 proteins that are known to be present in the cell. Importantly, it is uncertain, even with further advancements, whether MS will ever gain sufficient dynamic range to achieve single-cell protein sequencing in the future.

A number of technologies have been proposed for single-cell protein sequencing, and in general, their potential can be calibrated using these three metrics: (1) generality; (2) sensitivity; and (3) throughput. Generality pertains to whether the method can be used to identify arbitrary sequences of amino acids regardless of their chemical composition such as charge, hydrophobicity, and length. Generality also pertains to whether the method can distinguish native and PTM modified amino acids. Sensitivity relates to whether method holds the potential to ultimately measure a single amino acid in a single protein. Throughput pertains to whether the method holds the potential to ultimately sequence all proteins from a single human cell, where one human cell typically contains 10⁸-10⁹ protein molecules consisting of 10,000-20,000 different species.

Current technology development towards single-molecule protein sequencing broadly fall into three categories: 1) nanopore-based peptide fingerprinting, 2) Edman degradation-based peptide fluorescence fingerprinting; and 3) real-time dynamic protein sequencing.

Nanopore sequencing platforms use either pore-forming proteins or nanofabricated pores and measure the change in electric current as peptides translocate through the pore and create a “fingerprint” of the peptides. Theoretically, the strength of the nanopore sequencing platforms is that they can read a wide range of amino acids with high accuracy. For example, the aerolysin nanopore was shown to distinguish all 20 amino acids when they are located at the C-terminal of a polyarginine peptide. More recently, it has been demonstrated that MspA nanopores can be used to fingerprint peptides bearing single amino acid substitutions. In practice, however, the current implementations of nanopore sequencing are not general for sequencing arbitrary peptides. For example, the aforementioned studies were carried out only using highly charged model peptides, while the nonuniform charge of native peptides prohibit their translocation through the nanopores. Additionally, accurate identification of the peptide sequence at single amino acid resolution by a nanopore is exceptionally challenging because up to eight amino acids can contribute to the ionic current change when a peptide passes through a nanopore. Critically, perhaps the largest disadvantage of nanopore-based sequencing is its throughput. For example, it can require 30 minutes to measure a single 25 amino acid peptide. Given that a single cell contains ~10⁸ to 10⁹ peptides consisting of ~20,000 different species, even with massively parallel operation, it is uncertain whether nanopore sequencing can reach the throughput required for single-cell proteomics.

The second strategy, termed Edman degradation-based peptide fluorescence fingerprinting, combines Edman chemistry with single-molecule microscopy. In this approach, cysteine and lysine side chains are fluorescently labeled, and the peptides are immobilized on a solid support via the C-terminus. Next, the N-terminal amino acids are sequentially removed—one residue at a time—by Edman degradation. Digestion of fluorescently labeled amino acids causes decreases in fluorescence intensity, which serves as unique fingerprint for a given peptide. The strength of this method is that it potentially allows fingerprinting of millions of peptides in parallel, and that the approach is generalizable to most peptide sequences thanks to the robustness of Edman degradation. The key weakness of this method is that spectrally distinguishable fluorophores serve as the surrogates of amino acids, and thus specific labeling of amino acids with high efficiency is essential for this technique. Unfortunately, at this time, only fluorescent labeling of lysine and cysteine has been demonstrated. To date, only a small set of amino acids (e.g., lysine, cysteine, and tyrosine) can be labeled with sufficient specificity and efficiency required by this technique. Furthermore, photobleaching, chemical degradation of fluorophores, and energy transfer between fluorophores (in the context of a peptide) all likely limit the sensitivity and generalizability of this technique.

Finally, real-time dynamic protein sequencing utilizes continuous degradation of surface—immobilized peptides carried out by aminopeptidases. During the degradation, the nascent N-terminal amino acids are recognized in real-time by a mixture of dye-labeled N-terminal amino acid binders evolved from adaptor protein ClpS. N-terminal amino acids are identified not only based on binding affinity, but also by the binding kinetics, which allows identification of multiple amino acids by a single binder protein. The advantage of this method is that it is not restricted by the charge status of the peptides nor the chemical functionalities of amino acid side chains, and thus can be potentially generalizable to the majority of peptide sequences. In addition, the use of N-terminal amino acid binders also greatly expands the number of sequenceable amino acids. To date, the ClpS proteins used in real-time dynamic protein sequencing have been shown to distinguish seven different N-terminal amino acids. The key weakness of this strategy, however, is that the binding of ClpS proteins to N-terminal amino acids is affected by adjacent amino acids —which is a critical problem. Recently, it has been shown that that the affinity and binding kinetics of ClpS proteins varies drastically even within a small subset of possible downstream sequences. Thus, the feasibility of this methodology will hinge on the availability of novel reagents whose binding affinity and kinetics do not depend on amino acids that are connected to the N-terminal amino acid. Unfortunately, such reagents have not been discovered to date. Additionally, enzymatic degradation used in real-time dynamic sequencing does not proceed in a stepwise fashion. This leads to inconsistent lifetimes of ClpS-N-terminal amino acid complex and the lack of capability to measure the length of unidentified peptide segments, both of which limit the accuracy of this technique.

For the reasons set forth above, no current technology offers the generalizability, sensitivity and throughput to ultimately reach the goal of achieving single-cell protein sequencing.

### SUMMARY

Provided are methods of sequencing a polypeptide. In certain embodiments, the methods comprise labeling the N-terminal amino acid of a polypeptide with a nucleic acid label comprising a unique molecular identifier and cycle number barcode; degrading the N-terminal amino acid from the polypeptide; annealing a primer to the nucleic acid label of the degraded amino acid, where the primer comprises a barcode corresponding to the identity of the degraded N-terminal amino acid; and extending the primer annealed to the nucleic acid label to produce an extension product comprising the unique molecular identifier, the cycle number barcode, and the barcode corresponding to the identity of the degraded N-terminal amino acid. The preceding steps are performed successively to produce a plurality of such extension products which are then sequenced, enabling determination of the amino acid sequence of the polypeptide. Composition and kits that find use in practicing the methods are also provided.

### BRIEF DESCRIPTION OF THE FIGURES

FIG. 1A-1C: (1A) Overview of single-molecule polypeptide sequencing according to embodiments of the present disclosure. In this example, the approach combines the chemistry of Edman degradation with the massive parallelism of DNA sequencing-by-synthesis technology. (1B) Schematic illustration of embodiments in which the polypeptide is labeled with a nucleic acid label comprising a unique molecular identifier (UMI) and a cycle number barcode. (1C) Schematic illustration of embodiments in which biotinylated primers are employed, enabling the barcoded DNA to be pulled down by streptavidin beads on which binding-dependent primer extension occurs. Also in this example, the primer extension product is indexed with a cycle number barcode.

FIG. 2A-2D: (2A) Reaction scheme of intramolecular DNA encoded Edman degradation (INDEED) according to some embodiments of the present disclosure. In this example, polypeptides are conjugated to DNA unique molecular identifier (UMI) functionalized beads (Step 1). Primers used to record UMI are conjugated to the N-terminus of a polypeptide by modified PITC and click reaction (Steps 2 and 3). Then, the DNA UMI is transcribed by primer extension reaction (Step 4). Finally, the PTC amino acid is generated by cleavage and hydrolysis (Step 5). The remaining immobilized polypeptides are subjected to the next cycle of INDEED. (2B) Reaction scheme of binding-dependent primer extension (BD-PEX). A PTC amino acid is recognized by antibody tagged with primer and antibody-specific barcode (Step1). The binding event is recorded by primer extension. The resulting DNA are sequenced to reveal the polypeptide sequences (Step 2). (2C) Reaction scheme of INDEED according to some embodiments of the present disclosure. In this example, the C-terminal side of peptides are conjugated to DNA UMI functionalized beads (step 1). Subsequently, the N-terminus of the peptide is derivatized by azide-modified PITC, and a biotinylated primer is incorporated via a proximity-promoted click reaction (steps 2 and 3). In the fourth step, the UMIs are copied to nascent DNA strand after primer extension (step 4), Eventually, a Lewis acid-catalyzed cleavage, followed by hydrolysis under reductive basic conditions, releases the DNA-barcoded PTC amino acid from the solid support (steps 5 and 6). (2D) Scheme of antibody assisted proximity extension subsequent to the scheme described in 2C. DNA barcoded PTC amino acids are pulled down by streptavidin beads. Antibody tagged with primer and antibody specific barcode are introduced, and the recognition events are recorded by primer extension. The resultant DNA is made into sequencing libraries during which cycle number barcodes are introduced. This process generates DNA containing the information of position, origin and identity of amino acids and can be read by DNA sequencing.

FIG. 3A-3C: (3A) DNA modified by 7-deazapurine nucleotide is stable under the Lewis acid catalyzed Edman degradation condition. (3B) Reaction scheme of the Lewis acid catalyzed Edman degradation reaction. (3C) MS spectra of oligonucleotides containing 7-deazapurine deoxynucleotides after treatment with 40 mM BF3 etherate.

FIG. 4A-4C: (4A) Preparation of solid-phase immobilized DNA-peptide conjugate. (4B) Mass spectrum of PTC-tryptophan in supernatant released by Edman degradation on solid supports. Inset shows the chromatogram of the supernatant. (4C) Edman degradation on CPG. The DNA is cleaved from CPG by ammonia and analyzed by HPLC. PTC-peptide-DNA conjugate (0 min, right trace) was completely degraded to yield peptide-DNA conjugate (10 min, left trace).

FIG. 5A-5H: (5A) Synthesis of alkyne modified PITC derivative. 2. (5B) Preparation of DNA-peptide conjugate on beads and DNA barcoding of N-terminus amino acid by INDEED. (5C) Quantification of degradation yield by flow cytometry. Primer is hybridized with FAM-labeled complementary strand and quantified on flow cytometer. The decrease in fluorescence intensity after the degradation is used to calculate the degradation yield. (5D) Screening of polymerases for primer extension. Purine nucleotides in template and primer are fully substituted by 7-deazadA and 7-deazadG. dNTP mix contains dTTP, dCTP, 7-deazadATP, and 7-deazadGTP. (5E)-(5F) An embodiment of INDEED in which magnetic beads are employed and primers for barcode transfer are introduced via proximity promoted SPAAC. (5G) LC-MS data of DNA barcoded PTC amino acid generated by INDEED. (5H) Flow cytometry plot showing stepwise degradation yield and overall yield of INDEED process over five degradation cycles.

FIG. 6A-6F: (6A) Binding of commercially available antibodies to DNA conjugated PTC amino acids. (6B) Binding of phosphotyrosine specific antibody (PY20) to DNA conjugated PTC-phosphotyrosine. (6C) Specificity of antibodies generated by hybridoma raised against PTC-tyrosine conjugated BSA characterized by ELISA. (6D) Specificity of antibodies generated by hybridoma raised against PTC-phenylalanine conjugated BSA characterized by ELISA. (6E) Data demonstrating antibody recognition of PTC for asymmetric dimethylarginine (ADMA), acetyl lysine, and phosphoserine. (6F) Data demonstrating identification of antibodies against PTC modified phenylalanine, tyrosine, tryptophan, arginine and aspartic acid.

FIG. 7A-7E: (7A) Conversion of PTC amino acid to DNA by binding dependent primer extension (BD-PEX) according to some embodiments of the present disclosure. (7B) Conversion of PTC amino acid to DNA by BD-PEX according to some embodiments of the present disclosure. (7C) PAGE analysis of product generated by antibody assisted proximity extension. Primers with length of 5 nt and 6 nt can distinguish the presence of antibody-antigen interactions. (7D) Quantification of DNA output of antibody assisted proximity extension by qPCR. Primer with length of 6 nt gave quantitative DNA output and was chosen for future experiments. (7E) Reducing loading density on beads inhibited the occurrence of undesired intermolecular primer extension.

FIG. 8A-8B: Sequencing peptides by DNA sequencing. (8A) Scheme of sequencing single peptide species with multiple readable amino acids. The peptide was subjected to five cycles of INDEED process. The resulting DNA barcoded PTC amino acids from each cycle are pulled down onto streptavidin beads in separate containers. Proximity primer extension is carried out with a mixture of DNA barcoded PTC amino acid specific antibodies (100 nM of each of PTC-Arg antibody, PTC-Phe antibody, PTC-Asp antibody, PTC-Trp antibody). After primer extension, adaptor PCR are performed in each vessel with adaptor primers bearing cycle number barcode. Finally, all the DNA is pooled, indexed and sequenced on a MiSeq sequencer. (8B) Heatmap tallying the read count DNA barcodes obtained for peptide sequence RGFDW.

FIG. 9A-9C: Workflow of single-cell proteoform mapping. (9A) Single-cell peptide extraction. Single cells are isolated via FACS in multi-well plates containing lysis buffer. Proteins of interest are pulled down by antibody coated beads. These proteins are then digested by trypsin. (9B) Peptides are conjugated to DNA UMI that also include barcodes that are specific to each well. The barcode peptides are sequenced by INDEED. (9C) The distribution of proteoforms, for instance phosphorylation, can be mapped in each cell at single-amino acid resolution.

### DETAILED DESCRIPTION

Before the methods, compositions and kits of the present disclosure are described in greater detail, it is to be understood that the methods, compositions and kits are not limited to particular embodiments described, as such may, of course, vary. It is also to be understood that the terminology used herein is for the purpose of describing particular embodiments only, and is not intended to be limiting, since the scope of the methods, compositions and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, compositions and kits. The upper and lower limits of these smaller ranges may independently be included in the smaller ranges and are also encompassed within the methods, compositions and kits, subject to any specifically excluded limit in the stated range. Where the stated range includes one or both of the limits, ranges excluding either or both of those included limits are also included in the methods, compositions and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, compositions and kits belong. Although any methods, compositions and kits similar or equivalent to those described herein can also be used in the practice or testing of the methods, compositions and kits, representative illustrative methods, compositions and kits are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, compositions and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, compositions and kits, which are, for clarity, described in the context of separate embodiments, may also be provided in combination in a single embodiment. Conversely, various features of the methods, compositions and kits, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, compositions and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods of Sequencing Polypeptides

Aspects of the present disclosure include methods of sequencing polypeptides. According to some embodiments, the methods comprise labeling the N-terminal amino acid of a polypeptide with a nucleic acid label comprising a unique molecular identifier (UMI), and degrading the N-terminal amino acid from the polypeptide. In certain embodiments, such methods further comprise annealing a primer to the nucleic acid label of the degraded N-terminal amino acid, wherein the primer comprises a barcode corresponding to the identity of the degraded N-terminal amino acid. In some instances, such methods further comprise extending the primer annealed to the nucleic acid label to produce an extension product comprising the UMI and the barcode corresponding to the identity of the degraded N-terminal amino acid. The preceding steps may be performed in successive cycles to produce a plurality of extension products, each of the plurality of extension products comprising the UMI, a respective cycle number barcode, and a barcode corresponding to the identity of a respective degraded N-terminal amino acid. According to some embodiments, such methods further comprise sequencing the plurality of extension products, and determining a sequence of the polypeptide based on the sequences of the plurality of extension products. In certain embodiments, the methods comprise indexing the extension product produced at the extending step with the cycle number barcode. In other embodiments, the labeling step comprises labeling the N-terminal amino acid of the polypeptide with a nucleic acid label comprising the UMI and the cycle number barcode.

The methods of the present disclosure address the shortcomings and constitute an improvement over the nanopore-based polypeptide sequencing, Edman degradation-based peptide fluorescence fingerprinting, and real-time dynamic protein sequencing approaches. For example, nanopore-based polypeptide sequencing requires that the protein be charged to enable translocation, and additionally suffers from low throughput. Regarding Edman degradation-based peptide fluorescence fingerprinting, only fluorescent labeling of lysine and cysteine has been demonstrated to date. With respect to real-time dynamic protein sequencing, shortcomings include the effect of protein sequences on recognizer binding.

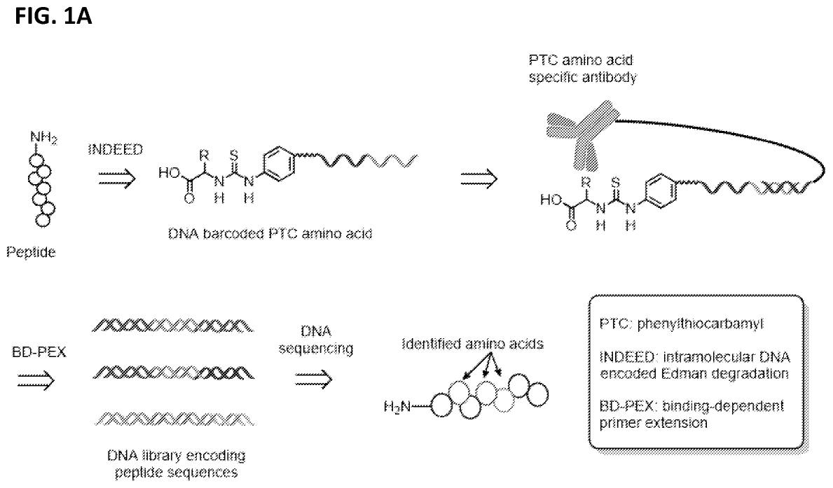

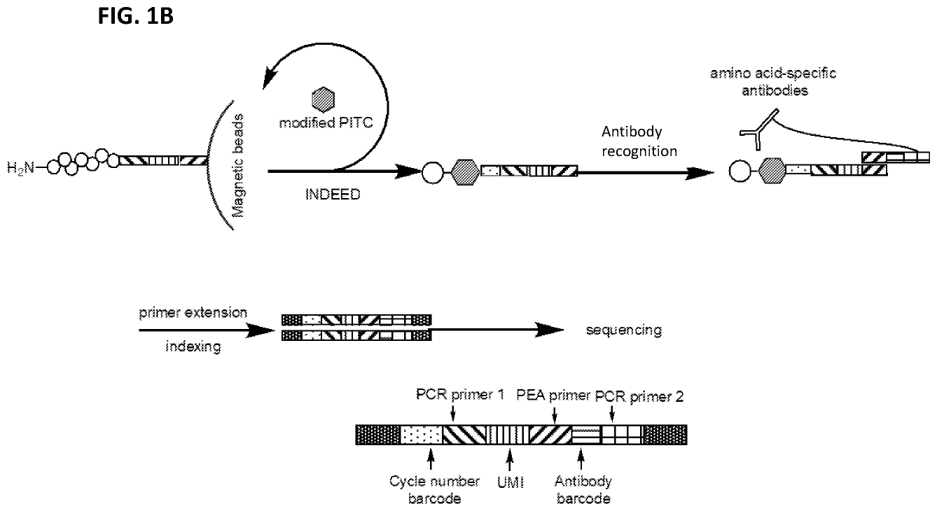

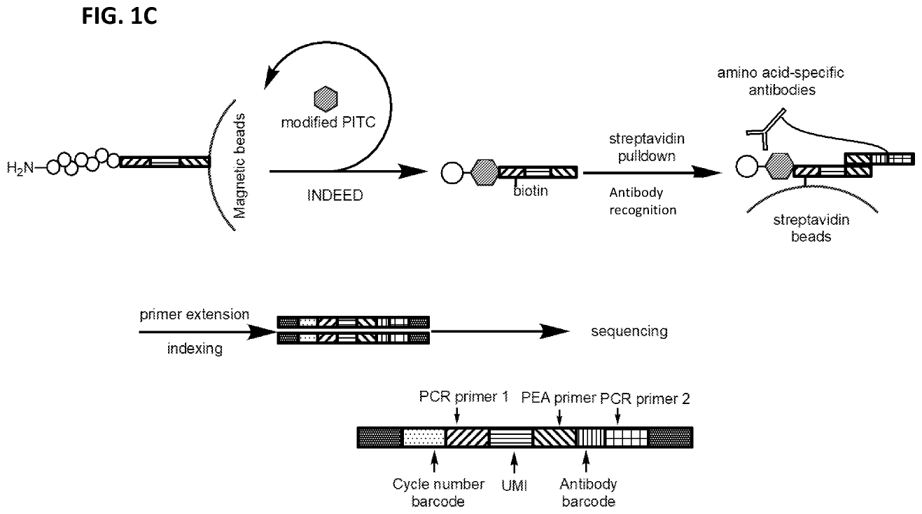

An overview of embodiments of the methods of the present disclosure is schematically illustrated in FIG. 1. In this example, Edman degradation is implemented to create a “degradation fragment” which is then labeled with a DNA barcode that encodes the origin of the amino acid (i.e., the polypeptide from which it came) and its position within the polypeptide. These fragments are specifically recognized by binding agents (e.g., antibodies, small molecules, aptamers, or the like) free of interference of their original downstream polypeptide sequences. The binding of the binding agent allows a DNA barcode to be linked to the amino acid, which enables the peptide sequence to be decoded in a massively parallel manner using a nucleic acid sequencer, e.g., an Illumina-style or other suitable DNA sequencer.

Thus, the methods of the present disclosure—embodiments of which are sometimes referred to herein as intramolecular DNA encoded Edman degradation (or “INDEED”)—comprise two key process modules. The first module degrades the terminal amino acid and generates DNA barcoded amino acids (e.g., phenylthiocarbamyl (PTC)-amino acids). The second module identifies the amino acids by proximity primer extension and reading polypeptide sequences by DNA sequencing.

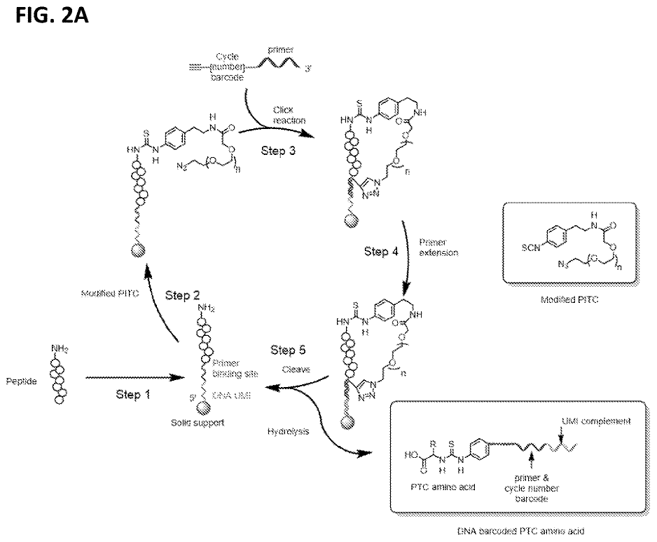

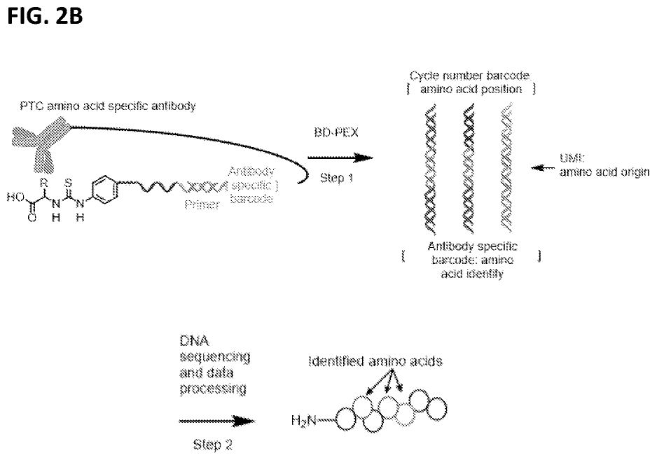

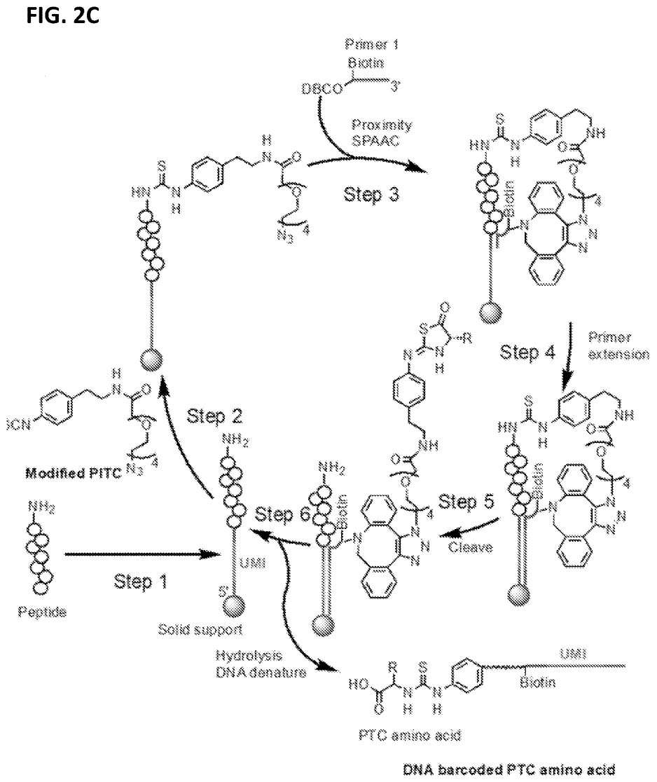

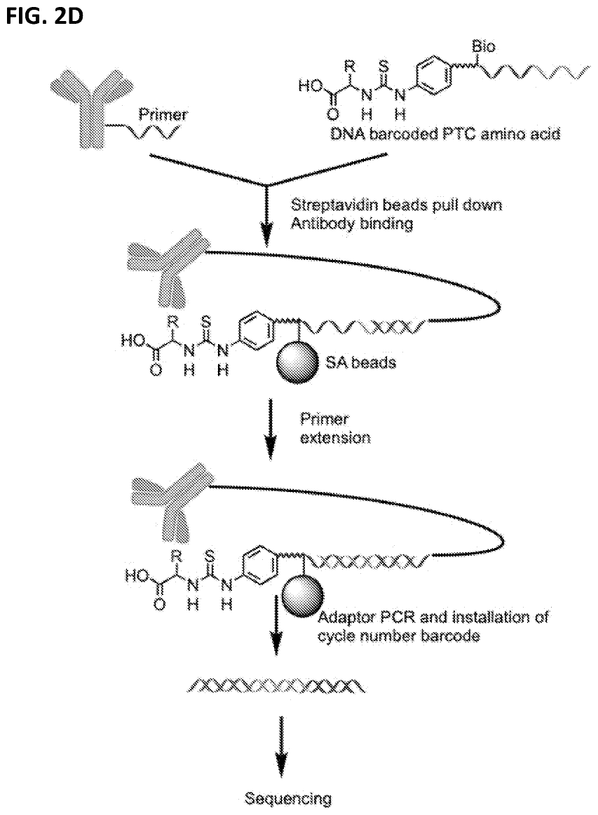

A non-limiting example of a first module according to embodiments of the present disclosure is schematically illustrated in FIG. 2A. In the first step, polypeptides are immobilized on solid supports (e.g., beads or other suitable solid supports). This may be achieved by conjugating the C-terminal region of polypeptides to DNA unique molecular identifier (UMI) functionalized beads so that each peptide is attached to a unique DNA sequence (FIG. 2A, step 1). Then, in the ensuing two steps, primers used to record the UMI are conjugated to the N-terminus of a polypeptide (FIG. 2A, step 2 and 3). This may be carried out by reacting the polypeptides with a modified isothiocyanate (e.g., a modified PITC) bearing a click handle. Subsequently, a primer containing the barcode for cycle number is installed via click chemistry. In the fourth step, the DNA UMI is transcribed by a proximity primer extension reaction (FIG. 2A, step 4). This step transfers the information of the parent polypeptide to the Edman degradation fragments. In the final step, the PTC amino acid is cleaved from the polypeptide (FIG. 2A, step 5). This step may be achieved by a cleavage-hydrolysis tandem reaction which generates a PTC amino acid fragment barcoded with DNA containing the information of the cycle number and the parent polypeptide. This process is made possible by a modified Edman degradation and the use of unnatural nucleotides, which allows the preservation of nucleic acids under the harsh conditions of polypeptide sequencing.

A non-limiting example of a second module according to embodiments of the present disclosure is schematically illustrated in FIG. 2B. In this example, the second module for reading out polypeptide sequences by DNA sequencing is carried out in two steps. First, identity information of the amino acid fragment is converted to a specific DNA sequence. To do so, PTC fragments are recognized by their corresponding binding agent (e.g., antibody, small molecule, aptamer, or the like) conjugated to a primer comprising a binding agent-specific barcode sequence. The binding events are recorded by binding-dependent primer extension (BD-PEX) (FIG. 2B, step 1). This process yields DNA duplexes containing the information of the parent polypeptides, and the order and identity of the amino acids. Second, polypeptide sequences are read by DNA sequencing (FIG. 2B, step 2). This is conducted by combining the DNA encoding polypeptide sequences generated by successive cycles and sequencing these DNA on a sequencing platform. The resulting DNA sequencing data is used for reconstruction of polypeptide sequences. This is accomplished by attributing DNA bearing the same UMI to a single parent polypeptide and assigning the order and identity of amino acids using cycle number barcodes and binding agent-specific barcodes.

Further non-limiting examples of modules according to embodiments of the present disclosure are schematically illustrated in FIGS. 2C-2D.

The advantages of the methods of the present disclosure over the existing methods for polypeptide sequencing are numerous. First, the methods are general. That is, the methods may implement the well-established Edman degradation which is compatible for polypeptide sequences with varying charges and lengths. The methods directly detect the degradation fragments, which are extracted from their sequence contexts and recognized by binding agents (e.g., antibodies). Affinity-based detection does not rely on the chemical properties of amino acids and is generalizable to all proteogenic amino acids and their post-translationally modified forms. Second, the methods allow sequencing of polypeptides at single amino acid resolution with single-molecule sensitivity. The methods take off one amino acid from the N-terminus each cycle and barcodes the resulting fragment with DNA encoding the origin and position of the amino acid. Readout of DNA barcoded PTC amino acids without the interference of downstream polypeptide sequences by BD-PEX enables polypeptide sequencing to be carried out at single amino acid resolution. The resulting DNA sequences are amplified during DNA sequencing, which enables sequencing of polypeptides with single-molecule sensitivity. Third, the methods enable the throughput required for single-cell protein sequencing. The methods achieve highly parallel Edman degradation by DNA barcoding and converts polypeptide sequence information into DNA sequences. Current DNA sequencing technology can already reach>10¹⁰ reads per run (e.g., using Illumina's NovaSeq® sequencing platform). Given that the throughput of DNA sequencing continues to increase, the throughput of present methods will reach that required to sequence all proteins from a single cell. Details regarding embodiments of the methods of the present disclosure will now be described.

The terms “polypeptide”, “peptide”, and “protein” are used interchangeably herein to designate a linear series of amino acid residues connected one to the other by peptide bonds between the alpha-amino and carboxy groups of adjacent residues. The amino acids may include the 20 “standard” genetically encodable amino acids, non-natural amino acids (e.g., amino acid analogs), or a combination thereof.

The term “amino acid” generally refers to any monomer unit that comprises a substituted or unsubstituted amino group, a substituted or unsubstituted carboxy group, and one or more side chains or groups, or analogs of any of these groups. Exemplary side chains include, e.g., thiol, seleno, sulfonyl, alkyl, aryl, acyl, keto, azido, hydroxyl, hydrazine, cyano, halo, hydrazide, alkenyl, alkynl, ether, borate, boronate, phospho, phosphono, phosphine, heterocyclic, enone, imine, aldehyde, ester, thioacid, hydroxylamine, or any combination of these groups. Naturally-occurring α-amino acids are those encoded by the genetic code as well as those amino acids that are later modified (e.g., hydroxyproline, γ-carboxyglutamate, and O-phosphoserine). Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (IIe), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Amino acids may be referred to herein by either their commonly known three letter symbols or by the one-letter symbols recommended by the IUPAC-IUB Commission on Biochemical Nomenclature. For example, an L-amino acid may be represented herein by its commonly known three letter symbol (e.g., Arg for L-arginine) or by an upper-case one-letter amino acid symbol (e.g., R for L-arginine). A D-amino acid may be represented herein by its commonly known three letter symbol (e.g., D-Arg for D-arginine) or by a lower-case one-letter amino acid symbol (e.g., r for D-arginine).

The polypeptide may be present in any sample of interest, including but not limited to, a protein sample isolated from a single cell, a plurality of cells (e.g., cultured cells), a tissue, a biological fluid (e.g., whole blood or a fraction thereof, urine, saliva, cerebrospinal fluid, sputum, etc.), an organ, or an organism (e.g., bacteria, yeast, or the like). In certain embodiments, the protein sample is isolated from a cell(s), tissue, organ, and/or the like of a mammal (e.g., a human, a rodent (e.g., a mouse), or any other mammal of interest). In other embodiments, the protein sample is isolated from a source other than a mammal, such as bacteria, yeast, insects (e.g., drosophila), amphibians (e.g., frogs (e.g., Xenopus)), viruses, plants, or any other non-mammalian protein sample source.

In certain embodiments, the polypeptide to be sequenced is present in a protein sample isolated from a single cell. In some such instances, the method is a single-cell protein sequencing method performed on a plurality of polypeptides present in the protein sample isolated from the single cell.

Approaches, reagents and kits for isolating proteins from single cells, cell populations, tissues, etc. are known in the art. Non-limiting examples of available protein extraction kits include the ReadyPrep™ Protein Extraction Kit (Bio-Rad), a Qproteome Protein Isolation Kit (Qiagen), T-PER™ Tissue Protein Extraction Reagent (Thermo Scientific), M-PER™ Mammalian Protein Extraction Reagent (Thermo Scientific), B-PER™ Complete Bacterial Protein Extraction Reagent (Thermo Scientific), Pierce Plant Total Protein Extraction Kit (Thermo Scientific), RIPA buffer with Triton™ X-100 (5×) (Thermo Scientific), and the like.

Protein samples used in the methods of the present disclosure may be collected by any convenient means. In some instances, useful cellular samples may be or may be derived from a biopsy. Biopsy tissues may be obtained from healthy or diseased cells or tissues, including e.g., cancer cells or tissues. Thus, in some embodiments, the biopsy sample is a tumor biopsy sample. Depending on the type of cancer and/or the type of biopsy performed the sample may be prepared from a solid tissue biopsy or a liquid biopsy.

In some instances, a protein sample may be prepared from a surgical biopsy. Any convenient and appropriate technique for surgical biopsy may be utilized for collection of a sample to be employed in the methods described herein including but not limited to, e.g., excisional biopsy, incisional biopsy, wire localization biopsy, and the like. In some instances, a surgical biopsy may be obtained as a part of a surgical procedure which has a primary purpose other than obtaining the sample, e.g., including but not limited to tumor resection, mastectomy, lymph node surgery, axillary lymph node dissection, sentinel lymph node surgery, and the like.

Various other biopsy techniques may be employed to obtain biopsy tissue, for use as a protein sample as described herein. As a non-limiting example, a sample may be obtained by a needle biopsy. Any convenient and appropriate technique for needle biopsy may be utilized for collection of a sample including but not limited to, e.g., fine needle aspiration (FNA), core needle biopsy, stereotactic core biopsy, vacuum assisted biopsy, and the like.

According to embodiments of the polypeptide sequencing methods of the present disclosure, the methods comprise labeling the N-terminal amino acid of a polypeptide with a nucleic acid label comprising a unique molecular identifier (UMI) and a cycle number barcode. As used herein in the context of the structure of a polypeptide, the “N-terminal amino acid” and “C-terminal amino acid” refer to the amino acid at the extreme amino and carboxyl ends of the polypeptide, respectively.

The term “unique molecular identifier (UMI)” or “UMI” as used herein refers to a sequence of nucleotides which can be used to identify and/or distinguish a first molecule to which the UMI is attached from one or more second molecules. As used herein, a UMI may include one or more nucleotides at one or both ends of the identifying/distinguishing sequence of nucleotides, e.g., to facilitate attachment (e.g., ligation) of the UMI to a different entity. UMIs are typically short, e.g., about 5 to 40 (e.g., about 5 to 20) bases in length. Generally, a UMI is used to distinguish between molecules of a similar type within a population or group.

As used herein, a “barcode” or “barcode sequence” refers to a uniquely identifiable nucleotide sequence. In some embodiments, a barcode uniquely identifies a degradation cycle number (cycle number barcode). Barcode sequences may vary widely in length and composition. According to some embodiments, the barcode has a degenerate sequence of from 4 to 120 nucleotides in length, e.g., from 4 to 100, 4 to 80, 4 to 60, 4 to 40, 6 to 30, 8 to 20 nucleotides, or 10 to 15 nucleotides in length. In certain embodiments, the barcode has a degenerate sequence of up to 20 nucleotides in length, e.g., 1, 2, 3, 4, 5, 6, 7, 8, 9, 10, 11, 12, 13, 14, 15, 16, 17, 18, 19 or 20 nucleotides in length. The barcode may include one or more mixed bases (e.g., every 3 bases, every 4 bases, or the like) of only three possible base combinations instead of four to prevent homopolymeric barcodes.

In certain embodiments, prior to the labeling step, the polypeptide is immobilized on a solid support via a nucleic acid attached to the C-terminus of the polypeptide and the surface of the solid support, wherein the nucleic acid comprises the UMI and a primer binding site 3′ or 5′ to the UMI. According to such embodiments, the labeling may comprise conjugating a degradation moiety to the N-terminal amino acid, and conjugating a primer to the degradation moiety, wherein the primer conjugated to the degradation moiety comprises the cycle number barcode and a sequence 3′ to the cycle number barcode which is complementary to the primer binding site of the nucleic acid immobilizing the polypeptide to the solid support. Such labeling may further comprise annealing the primer conjugated to the degradation moiety to the primer binding site, and extending the primer conjugated to the degradation moiety using the nucleic acid immobilizing the polypeptide to the solid support as the template, thereby labeling the N-terminal amino acid with the nucleic acid label comprising the UMI and the cycle number barcode.

As described above, according to some embodiments, the polypeptide is immobilized on a solid support via a nucleic acid attached to the C-terminus of the polypeptide and the surface of the solid support, wherein the nucleic acid comprises the UMI and a primer binding site 3′ or 5′ to the UMI. According to such embodiments, the labeling may comprise conjugating to the N-terminal amino acid a degradation moiety conjugated to a primer comprising the cycle number barcode and a sequence 3′ to the cycle number barcode which is complementary to the primer binding site of the nucleic acid immobilizing the polypeptide to the solid support. Such labeling may further comprise annealing the primer conjugated to the degradation moiety to the primer binding site, and extending the primer conjugated to the degradation moiety using the nucleic acid immobilizing the polypeptide to the solid support as the template, thereby labeling the N-terminal amino acid with the nucleic acid label comprising the UMI and the cycle number barcode.

The term “solid support” means an insoluble material having a surface to which reagents and/or materials (e.g., the polypeptide) can be directly or indirectly attached. In certain embodiments, a collection of solid supports has an average greatest dimension of 750 μm or less, 500 μm or less, 250 μm or less, 100 μm or less, 1 μm or less, 0.75 μm or less, 0.50 μm or less, 0.25 μm or less, or 0.1 μm or less.

A variety of materials can be used as solid supports. Support materials include any material that can act as a support for attachment of the reagents and/or materials. Suitable materials include, but are not limited to, organic or inorganic polymers, natural and synthetic polymers, including, but not limited to, agarose, cellulose, nitrocellulose, cellulose acetate, other cellulose derivatives, dextran, dextran-derivatives and dextran co-polymers, other polysaccharides, glass, silica gels, gelatin, polyvinyl pyrrolidone, rayon, nylon, polyethylene, polypropylene, polybutylene, polycarbonate, polyesters, polyamides, vinyl polymers, polyvinylalcohols, polystyrene and polystyrene copolymers, polystyrene cross-linked with divinylbenzene or the like, acrylic resins, acrylates and acrylic acids, acrylamides, polyacrylamides, polyacrylamide blends, co-polymers of vinyl and acrylamide, methacrylates, methacrylate derivatives and co-polymers, other polymers and co-polymers with various functional groups, latex, butyl rubber and other synthetic rubbers, silicon, glass, paper, natural sponges, insoluble protein, surfactants, metals, metalloids, magnetic materials, and any combinations thereof.

The solid supports may be any suitable shape, including but not limited to spherical, spheroid, rod-shaped, disk-shaped, pyramid-shaped, cube-shaped, cylinder-shaped, nanohelical-shaped, nanospring-shaped, nanoring-shaped, arrow-shaped, teardrop-shaped, tetrapod-shaped, prism-shaped, or any other suitable geometric or non-geometric shape.

In certain embodiments, the solid supports are beads. As used herein, the term “bead” refers to a small mass that is generally spherical or spheroid in shape. According to some embodiments, a bead as used herein has an average diameter of from about 0.50 μm to about 500 μm, e.g., from about 0.75 μm to about 250 μm, e.g., about 1 μm.

Additionally, and for purposes herein, solid supports may be magnetically responsive, e.g., by virtue of comprising one or more paramagnetic and/or superparamagnetic substances, such as for example, magnetite. Such paramagnetic and/or superparamagnetic substances may be embedded within the matrix of the solid supports, and/or may be disposed on an external and/or internal surface of the solid support, e.g., bead.

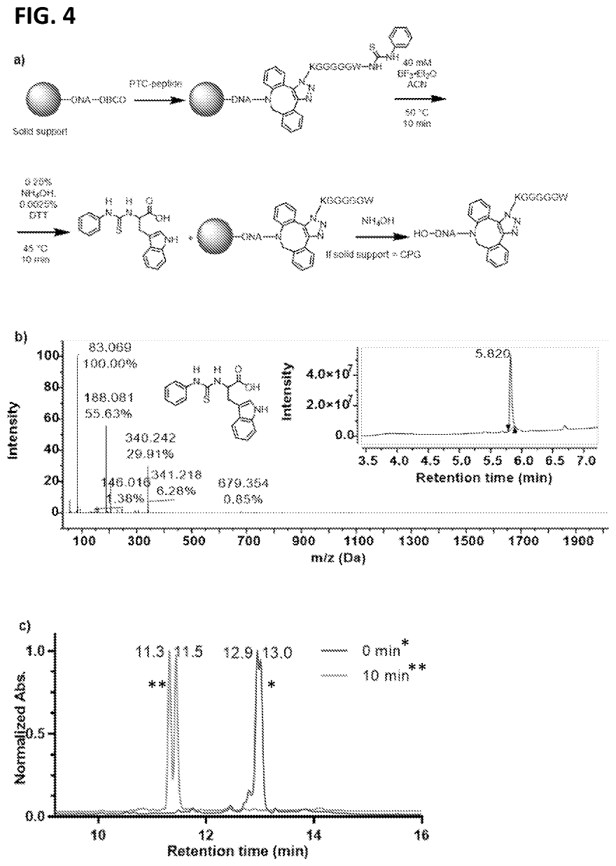

A variety of approaches may be employed to immobilize the polypeptide to a solid support, including those described in detail in the Experimental section herein. For example, in some instances, a dibenzocyclooctyne (DBCO) modified DNA sequence is synthesized on controlled pore glass (CPG) or polystyrene coated carboxylic magnetic beads. CPGs are commonly used for solid-phase DNA synthesis, and magnetic beads are generally compatible with enzymes and allow easy separation. In one non-limiting embodiment, a PTC-peptide containing a C-terminal azidolysine may be conjugated with DBCO modified DNA via a strain-promoted alkyne-azide cycloaddition (SPAAC) to form a model DNA-peptide conjugate. See, e.g., FIG. 4A.

As demonstrated in the Experimental section herein, the inventors have determined that Edman degradation can be performed on an immobilized DNA-peptide conjugate. However, as DNA is unstable under traditional Edman degradation conditions, implementation of alternative Edman degradation reaction conditions compatible with DNA was required. Suitable alternative Edman degradation reaction conditions identified by the inventors include, but are not limited to, a Lewis acid in an aprotic solvent. In some instances, the Lewis acid is BF₃ etherate, BCI₃, BBr₃, Scandium(III) triflate, or any combination thereof. For example, the Lewis acid may comprise or consist of BF₃ etherate. According to some embodiments, the aprotic solvent is acetonitrile, N,N-dimethylformamide (DMF), dimethyl sulfoxide (DMSO), or any combination thereof. In one non-limiting example, the alternative Edman degradation reaction conditions comprise BF₃ etherate in an aprotic solvent, e.g., 40 mM BF₃ etherate in anhydrous acetonitrile. Suitable alternatives further include triethylamine acetate in dimethylformamide (DMF), e.g., at 70° C.

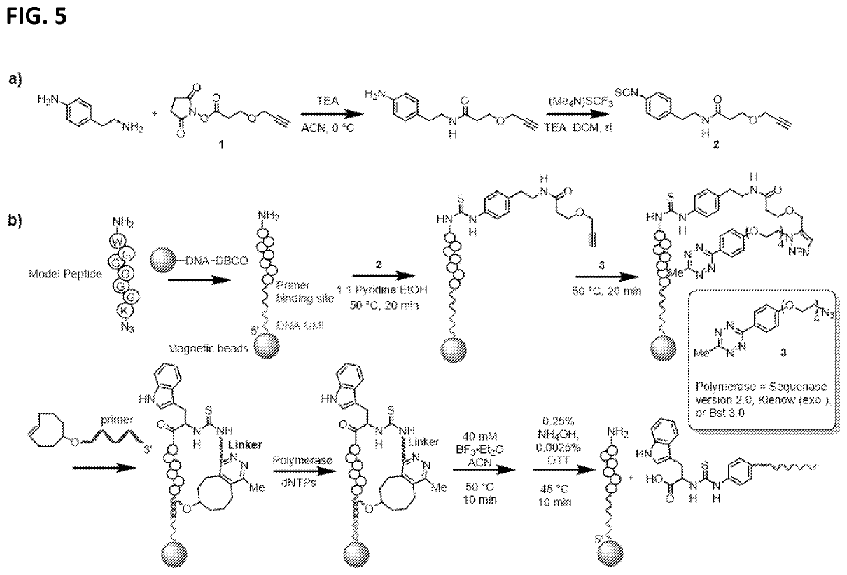

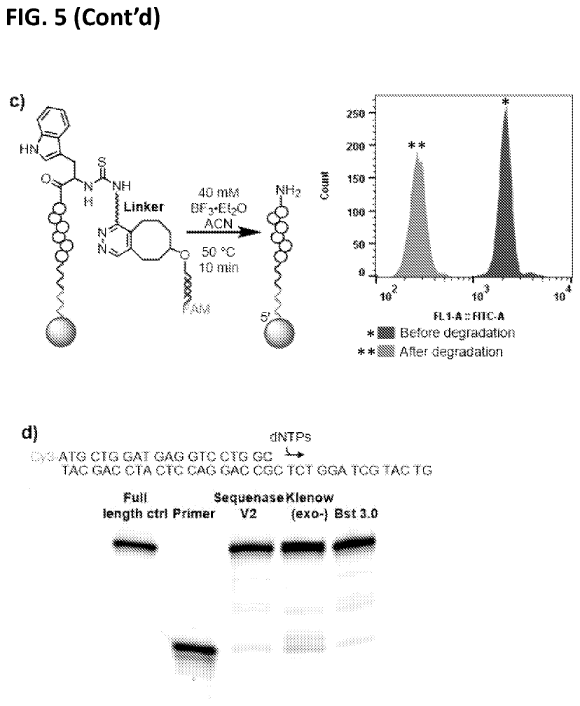

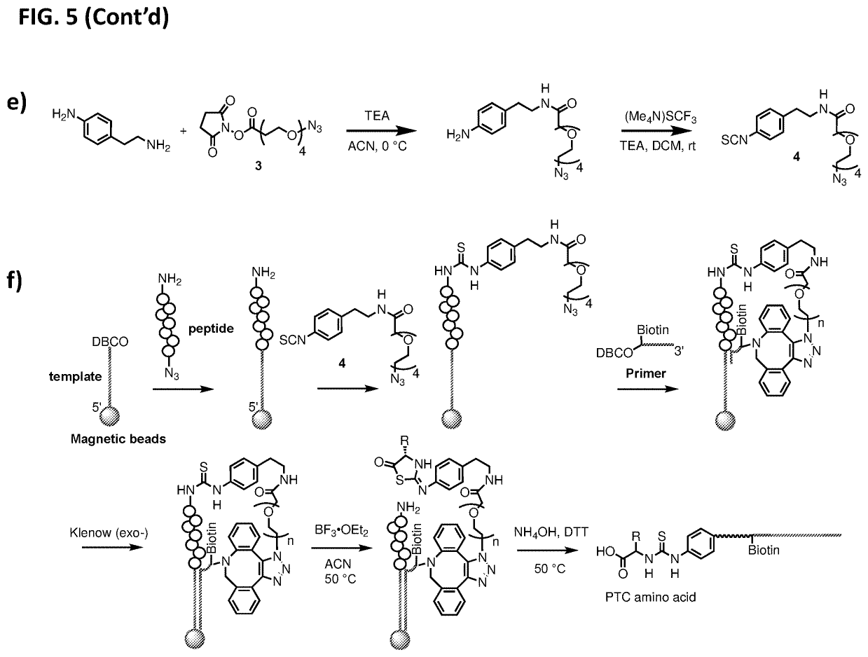

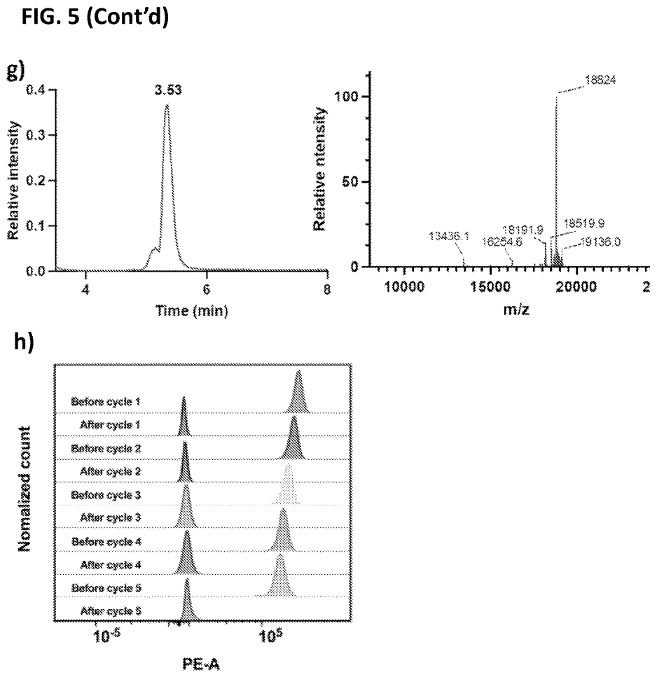

To further enhance the stability of nucleic acids implemented in the methods, one or more stability-enhancing non-natural nucleotides may be employed in any of the DNAs utilized in the methods. According to some embodiments, one or more of the DNAs utilized in the methods comprise one or more thermostability-increasing nucleotides. Non-limiting examples of thermostability-increasing nucleotides include 7-deaza-8-aza-purine-Triphoshpate, 2-Amino-2′-deoxyadenosine-5′-Triphosphate (2-Amino-dATP), 5-Methyl-2′-deoxycytidine-5′-Triphosphate (5-Me-dCTP), 5-Propynyl-2′-deoxycytidine-5′-Triphosphate (5-Pr-dCTP), 5-Propynyl-2′-deoxyuridine-5′-Triphosphate (5-Pr-dUTP) and or halogenated deoxy-uridine (XdU) like 5-Chloro-2′-deoxyuridine-5′-Triphosphate (5-Cl-dUTP), 5-Bromo-2′-deoxyuridine-5′-Triphosphate (5-Br-dUTP), and any combinations thereof. In certain embodiments, one or more nucleic acids comprise non-natural nucleotides that stabilize the nucleic acids during the degrading step, a non-limiting example of which are 7-deazapurine nucleotides. For example, the present disclosure surprisingly demonstrates that polymerases (e.g., Sequenase version 2.0, Klenow (exo-), and Bst 3.0) are able to accept 7-deazapurine nucleotide substituted template-primer duplexes and 7-deazapurine nucleoside triphosphates as substrates. See, e.g., Example 3 in the Experimental section below, and FIG. 5D.

In certain embodiments, the methods comprise conjugating a degradation moiety to the N-terminal amino acid. By “degradation moiety” is meant a moiety which, when conjugated to the N-terminal amino acid of a polypeptide, facilitates the cleavage of the N-terminal amino acid from the polypeptide under conditions compatible with the degradation moiety. In one non-limiting example, the degradation moiety employed is an isothiocyanate (ITC). Non-limiting examples of ITCs which may be employed as degradation moieties when practicing the methods of the present disclosure include phenylisothiocyanate (PITC), a substituted phenylisothiocyanate (e.g., para-substituted phenylisothiocyanate, ortho-substituted phenylisothiocyanate, meta-substituted phenylisothiocyanate, pentafluoro phenylisothiocyanate, and the like), alkyl isothiocyanate, naphthalenyl isothiocyanate, and the like. The term “conjugation” or “conjugating” generally refers to a chemical linkage, either covalent or non-covalent, usually covalent, that proximally associates one molecule of interest with a second molecule of interest.

According to some embodiments, the methods comprise conjugating a primer to the degradation moiety, where the primer conjugated to the degradation moiety comprises the cycle number barcode and a sequence 3′ to the cycle number barcode which is complementary to the primer binding site of the nucleic acid immobilizing the polypeptide to the solid support. A variety of approaches may be employed to conjugate a primer to the degradation moiety. In one non-limiting example, the degradation moiety comprises an isothiocyanate (e.g., PITC or the like) bearing a reactive group for conjugation to the nucleic acid comprising the cycle number barcode. In certain embodiments, the reactive group is a click-chemistry reactive group.

Click-chemistry reactions that may be employed include (i) nucleophilic substitutions; (ii) additions to C-C multiple bonds (e.g., Michael addition, epoxidation, dihydroxylation, aziridination); (iii) nonaldol like chemistry (e.g., N-hydroxysuccinimide active ester couplings); and (iv) cycloadditions (e.g., Diels-Adler reaction, Huisgen's cycloaddition). Huisgen's cycloaddition has been applied in various branches of chemistry. It consists of the condensation of organic azides with alkyne groups to form 1,2,3-triazole linkages. Azide and alkyne functionalities can be easily introduced in the scaffold of large organic constructs of biological relevance. The reaction may be catalyzed by introducing copper(I). The Cu(I) core has a dual effect in that it activates the slow-reacting alkyne group thus accelerating the azide-alkyne condensation kinetics by ~10⁷-10⁸-fold, and it organizes the reacting groups by “templation” so that only a regiospecific 1,4-disubstituted adduct is formed. This reaction is known as the copper-catalyzed azide alkyne cycloaddition (CuAAC), and its compatibility with a wide range of biological substrates and synthetic conditions makes CuAAC the flagship among click conjugations. Since its discovery, Cu(I)-catalyzed azide alkyne cycloaddition has been widely used within the fields of biology, biochemistry, and biotechnology. Click-chemistry reactions that may be employed include, but are not limited to, Huisgen Azide-Alkyne 1,3-Dipolar Cycloaddition, Copper-Catalyzed Azide-Alkyne Cycloaddition (CuAAC), Ruthenium-Catalyzed Azide-Alkyne Cycloaddition (RuAAC), and the like. Details regarding click-chemistry with nucleic acids are found, e.g., in Fantoni et al. (2021) Chem. Rev. 121(12):7122-7154.

The methods of the present disclosure include one or more annealing steps, e.g., annealing a primer conjugated to a degradation moiety to a primer binding site of a nucleic acid immobilizing the polypeptide to the solid support, annealing a primer to the nucleic acid label of the degraded N-terminal amino acid, and/or the like. One of ordinary skill in the art may design the various nucleic acids employed in the methods of the present disclosure so that they are capable of annealing to each other as desired, e.g., by designing the nucleic acids to have complementary regions as appropriate. The terms “complementary” or “complementarity” as used herein refer to a nucleotide sequence of a first nucleic acid that base-pairs by non-covalent bonds to a region of a second nucleic acid, or a nucleotide sequence of a first region of a nucleic acid that base-pairs by non-covalent bonds to a second region of the nucleic acid (e.g., a stem region). In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” or “complementarity” refers to a nucleotide sequence that is at least partially complementary. These terms may also encompass duplexes that are fully complementary such that every nucleotide in one strand is complementary to every nucleotide in the other strand in corresponding positions. In certain cases, a nucleotide sequence may be partially complementary to a target, in which not all nucleotides are complementary to every nucleotide in the target nucleic acid in all the corresponding positions. For example, a region of a first nucleic acid may be perfectly (i.e., 100%) complementary to a region of a second nucleic acid, or the region of the first nucleic acid may share some degree of complementarity which is less than perfect (e.g., 70%, 75%, 85%, 90%, 95%, 99%). The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=#of identical positions/total #of positions×100). When a position in one sequence is occupied by the same nucleotide as the corresponding position in the other sequence, then the molecules are identical at that position. A non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In some embodiments, parameters for sequence comparison can be set at score=100, wordlength=12, or can be varied (e.g., wordlength=5 or wordlength=20).

The conditions during an annealing step may be those conditions in which a first nucleic acid (e.g., primer) specifically hybridizes to a second nucleic acid (e.g., template nucleic acid). Whether specific hybridization occurs is determined by such factors as the degree of complementarity between the relevant portions of the nucleic acids, the length thereof, and the temperature at which the hybridization occurs, which may be informed by the melting temperatures (T_{M}) of the relevant portion of the nucleic acid. The melting temperature refers to the temperature at which half of the nucleic acids remain hybridized and half of the nucleic acids dissociate into single strands. The Tm of a duplex may be experimentally determined or predicted using the following formula Tm=81.5+16.6(log 10[Na⁺])+0.41 (fraction G+C)−(600/N), where N is the chain length and [Na⁺] is less than 1 M. See Sambrook and Russell (2001; Molecular Cloning: A Laboratory Manual, 3rd ed., Cold Spring Harbor Press, Cold Spring Harbor N. Y., Ch. 10). Other more advanced models that depend on various parameters may also be used to predict Tm of nucleic acid duplexes depending on various hybridization conditions. Approaches for achieving specific nucleic acid hybridization may be found in, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993).

In certain embodiments, the polypeptide sequencing methods of the present disclosure comprise annealing a primer to the nucleic acid label of the degraded N-terminal amino acid, where the primer comprises a barcode corresponding to the identity of the degraded N-terminal amino acid. In one non-limiting embodiment, at this annealing step, the primer comprising the barcode corresponding to the identity of the degraded N-terminal amino acid is conjugated to a binding moiety that specifically binds the degraded N-terminal amino acid, and wherein the annealing is dependent upon binding of the binding moiety to the degraded N-terminal amino acid.

A variety of suitable binding moieties may be employed, non-limiting examples of which include polypeptide binding moieties (e.g., antibodies), small molecules, aptamers, and the like. According to some embodiments, the binding moiety is an antibody. The term “antibody” may include an antibody or immunoglobulin of any isotype (e.g., IgG (e.g., IgG1, lgG2, lgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the cell surface molecule of the target cell, including, but not limited to single chain Fv (scFv), Fab, (Fab′)₂, (scFv′)₂, and diabodies; chimeric antibodies; monoclonal antibodies, human antibodies, humanized antibodies (e.g., humanized whole antibodies, humanized half antibodies, or humanized antibody fragments, e.g., humanized scFv); and fusion proteins comprising an antigen-binding portion of an antibody and a non-antibody protein. According to some embodiments, the antibody is selected from an IgG, Fv, single chain antibody, scFv, Fab, F(ab′)2, or Fab′. In certain embodiments, the antibody is a nanobody (an antibody fragment consisting of a single monomeric variable antibody domain—also known as a single-domain antibody (sdAb)), a monobody (a synthetic binding protein constructed using a fibronectin type III domain (FN3) as a molecular scaffold), or a Bi-specific T-cell engager (BiTE).

An immunoglobulin light or heavy chain variable region (V_{L} and V_{H}, respectively) is composed of a “framework” region (FR) interrupted by three hypervariable regions, also called “complementarity determining regions” or “CDRs”. The extent of the framework region and CDRs have been defined (see, E. Kabat et al., Sequences of proteins of immunological interest, 4th ed. U.S. Dept. Health and Human Services, Public Health Services, Bethesda, MD (1987); and Lefranc et al. IMGT, the international ImMunoGene Tics information system®. Nucl. Acids Res., 2005, 33, D593-D597)). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

An “antibody” thus encompasses a protein having one or more polypeptides that can be genetically encodable, e.g., by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, yeast or phage clone, or produced via a cell-free expression system, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, yeast display technologies, phage display technologies, ribosome display technologies, DNA display technologies, and the like. For example, monoclonal antibodies may be made by the hybridoma method first described by Kohler et al, Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature 352:624-628 (1991) and Marks et al, J. Mol. Biol. 222:581-597 (1991), for example.

According to some embodiments, the binding moiety is a small molecule. By “small molecule” compound is meant a compound having a molecular weight of 1000 atomic mass units (amu) or less. In some embodiments, the small molecule is 900 amu or less, 750 amu or less, 500 amu or less, 400 amu or less, 300 amu or less, or 200 amu or less. In some instances, the small molecule is not made of repeating molecular units such as are present in a polymer.

In certain embodiments, the binding moiety is an aptamer. By “aptamer” is meant a nucleic acid (e.g., an oligonucleotide) that has a specific binding affinity for the target cell surface molecule. Aptamers exhibit certain desirable properties, such as ease of selection and synthesis, high binding affinity and specificity, and versatile synthetic accessibility.

The phrases “specifically binds”, “specific for”, “immunoreactive” and “immunoreactivity”, and “antigen binding specificity”, when referring to a binding moiety (e.g., antibody, small molecule, aptamer, etc.), refer to a binding reaction with an antigen (e.g., a particular amino acid or post-translationally modified form thereof) which is highly preferential to the antigen, so as to be determinative of the presence of and/or selective for the antigen in the presence of a heterogeneous population of antigens (e.g., a mixture of different amino acids). Thus, under designated conditions, the specified binding moiety binds to a particular antigen and does not bind in a significant amount to other antigens present in the sample. Specific binding to an antigen under such conditions may require a binding moiety that is selected for its specificity for a particular antigen. For example, a binding moiety (e.g., an antibody) can specifically bind to a particular amino acid and does not exhibit comparable binding (e.g., does not exhibit detectable binding) to other amino acid present in a sample.

In some embodiments, a binding moiety “specifically binds” a particular amino acid if it binds to or associates with the particular amino acid with an affinity or Kₐ (that is, an equilibrium association constant of a particular binding interaction with units of 1/M) of, for example, greater than or equal to about 10⁵ M⁻¹ . In certain embodiments, the binding moiety binds to the particular amino acid with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10⁻⁵ M to 10⁻¹³ M, or less). In some embodiments, specific binding means the binding moiety binds to the particular amino acid with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the binding moiety for the particular amino acid can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

In certain embodiments, the binding moiety specifically binds a degraded N-terminal amino acid comprising a post-translational modification (PTM), and wherein the barcode indicates the identity of the degraded N-terminal amino acid and the PTM. PTMs are chemical modifications that play a key role in functional proteomic because they regulate activity, localization, and interaction with other cellular molecules such as proteins, nucleic acids, lipids and cofactors. PTMs of interest include, but are not limited to, phosphorylation, glycosylation, ubiquitination, nitrosylation, methylation, acetylation, or lipidation.

Protein phosphorylation, principally on serine, threonine or tyrosine residues, is one of the most important and well-studied post-translational modifications. Phosphorylation plays critical roles in the regulation of many cellular processes, including cell cycle, growth, apoptosis and signal transduction pathways. Protein glycosylation is acknowledged as one of the major post-translational modifications, with significant effects on protein folding, conformation, distribution, stability and activity. Glycosylation encompasses a diverse selection of sugar-moiety additions to proteins that ranges from simple monosaccharide modifications of nuclear transcription factors to highly complex branched polysaccharide changes of cell surface receptors. Carbohydrates in the form of asparagine-linked (N-linked) or serine/threonine-linked (O-linked) oligosaccharides are major structural components of many cell surface and secreted proteins. Ubiquitin is an 8-kDa polypeptide consisting of 76 amino acids that is appended to the ε-NH2 of lysine in target proteins via the C-terminal glycine of ubiquitin. Following an initial monoubiquitination event, the formation of a ubiquitin polymer may occur, and polyubiquitinated proteins are then recognized by the 26S proteasome that catalyzes the degradation of the ubiquitinated protein and the recycling of ubiquitin. S-nitrosylation is a critical PTM used by cells to stabilize proteins, regulate gene expression and provide NO donors, and the generation, localization, activation and catabolismof SNOs are tightly regulated. S-nitrosylation is a reversible reaction, and SNOs have a short half-life in the cytoplasm because of the host of reducing enzymes, including glutathione (GSH) and thioredoxin, that denitrosylate proteins. Therefore, SNOs are often stored in membranes, vesicles, the interstitial space and lipophilic protein folds to protect them from denitrosylation. The transfer of one-carbon methyl groups to nitrogen or oxygen (N-and O-methylation, respectively) to amino acid side chains increases the hydrophobicity of the protein and can neutralize a negative amino acid charge when bound to carboxylic acids. Methylation is mediated by methyltransferases, and S-adenosyl methionine (SAM) is the primary methyl group donor. N-acetylation, or the transfer of an acetyl group to nitrogen, occurs in almost all eukaryotic proteins through both irreversible and reversible mechanisms. N-terminal acetylation requires the cleavage of the N-terminal methionine by methionine aminopeptidase (MAP) before replacing the amino acid with an acetyl group from acetyl-CoA by N-acetyltransferase (NAT) enzymes. This type of acetylation is co-translational, in that N-terminus is acetylated on growing polypeptide chains that are still attached to the ribosome. 80 to 90% of eukaryotic proteins are acetylated in this manner. Lipidation is a method to target proteins to membranes in organelles (endoplasmic reticulum [ER], Golgi apparatus, mitochondria), vesicles (endosomes, lysosomes) and the plasma membrane. The four types of lipidation are: C-terminal glycosyl phosphatidylinositol (GPI) anchor; N-terminal myristoylation; S-myristoylation; and S-prenylation. Each type of modification gives proteins distinct membrane affinities, although all types of lipidation increase the hydrophobicity of a protein and thus its affinity for membranes. The different types of lipidation are also not mutually exclusive, in that two or more lipids can be attached to a given protein.

As summarized above, the labeling, degrading, annealing and extending steps are performed in successive cycles to produce a plurality of extension products, each of the plurality of extension products comprising the UMI, a respective cycle number barcode, and a barcode corresponding to the identity of a respective degraded N-terminal amino acid. Once the plurality of extension products are produced, the methods further comprise sequencing the plurality of extension products. As will be appreciated upon review of the present disclosure, the sequence of the polypeptide may be determined based on the sequences of the plurality of extension products.

Sequencing the plurality of extension products may be performed using any of a variety of available high throughput nucleic acid sequencers and systems. Illustrative sequencing systems include the Illumina iSeq 100, Miniseq, MiSeq series, NextSeq series (e.g., NextSeq 500 series, NextSeq 1000, NextSeq 2000), and NovaSeq sequencing systems (Illumina, Inc., San Diego, Calif.), the Pacific Biosciences Sequel (e.g., Sequel II) sequencing system (Pacific Biosciences, Menlo Park, Calif.), the Oxford Nanopore Technologies MinION™, GridIONx5™, PromethION™, or SmidgION™ nanopore-based sequencing systems (Oxford Nanopore Technologies, Oxford, UK), and other systems having similar capabilities.

In the Illumina platform, the sequencing process involves clonal amplification of adaptor-ligated DNA fragments on the surface of a glass slide. Bases are read using a cyclic reversible termination strategy, which sequences the template strand one nucleotide at a time through progressive rounds of base incorporation, washing, imaging, and cleavage. In this strategy, fluorescently labeled 3′-O-azidomethyl-dNTPs are used to pause the polymerization reaction, enabling removal of unincorporated bases and fluorescent imaging to determine the added nucleotide. Following scanning of the flow cell with a coupled-charge device (CCD) camera, the fluorescent moiety and the 3′ block are removed, and the process is repeated.

In zero mode waveguide (ZMW)-based sequence analysis, the ZMW is a nanoscale-sized well that serves as an optical confinement that allows observation of individual polymerase molecules. As a result, nucleotide incorporation events provide observation of an incorporating nucleotide analog that is readily distinguishable from non-incorporated nucleotide analogs. For a description of ZMWs and their application in nucleic acid sequencing, see, e.g., U.S. Patent Application Publication No. 2003/0044781 and U.S. Pat. No. 6,917,726, each of which is incorporated herein by reference in its entirety for all purposes. See also Levene et al. (2003) “Zero-mode waveguides for single-molecule analysis at high concentrations” Science 299:682-686, Eid et al. (2009) “Real-time DNA sequencing from single polymerase molecules” Science 323:133-138, and U.S. Pat. Nos. 7,056,676, 7,056,661, 7,052,847, 7,033,764, and 7,907,800, the full disclosures of which are incorporated herein by reference in their entirety for all purposes.

In nanopore sequencing, the nanopore serves as a biosensor and provides the sole passage through which an ionic solution on the cis side of the membrane contacts the ionic solution on the trans side. A constant voltage bias (trans side positive) produces an ionic current through the nanopore and drives ssDNA or ssRNA in the cis chamber through the pore to the trans chamber. A processive enzyme (e.g., a helicase, polymerase, nuclease, or the like) may be bound to the polynucleotide such that its step-wise movement controls and ratchets the nucleotides through the small-diameter nanopore, nucleobase by nucleobase. Because the ionic conductivity through the nanopore is sensitive to the presence of the nucleobase's mass and its associated electrical field, the ionic current levels through the nanopore reveal the sequence of nucleobases in the translocating strand. A patch clamp, a voltage clamp, or the like, may be employed.

Details for obtaining raw sequencing reads of nucleic acid molecules using nanopores are described, e.g., in Feng et al. (2015) Genomics, Proteomics & Bioinformatics 13(1):4-16. Nanopore-based sequencing systems are available and include the SmidgION, MinION, GridION, and PromethION nanopore-based sequencing systems available from Oxford Nanopore Technologies Limited. Detailed design considerations and protocols for performing nucleic acid sequencing are provided with such systems.

The methods of the present disclosure may be performed in any suitable container/confinement. One or more steps of the methods may be performed in a first container while one or more other steps are performed in a second container. Non-limiting examples of containers in which one or more steps of the methods may be performed include a tube, vial, plate, a well of a multi-well plate (e.g., a 6-, 12-, 24-, 48-, 96-or 384-well plate), a confinement within a microfluidic device, etc.

### Compositions and Kits

Aspects of the present disclosure further include compositions. In some embodiments, provided is a composition comprising one or more of any of polypeptides and/or one or any combination of reagents for performing the polypeptide sequencing methods of the present disclosure described elsewhere herein. Non-limiting examples of reagents are combinations thereof which may be present in a composition of the present disclosure include UMI-functionalized solid supports, a degradation moiety bearing a reactive group for conjugation to a nucleic acid, a primer comprising a cycle number barcode, Edman degradation reagents (including those for providing the alternative DNA compatible Edman degradation conditions described elsewhere herein), a conjugate comprising a primer conjugated to a binding moiety that specifically binds an amino acid, a conjugate comprising a primer conjugated to a binding moiety that specifically binds an amino acid bearing a post-translational modification, nucleic acid sequencing adapters, and any combination thereof.

According to some embodiments, a composition of the present disclosure comprises any of polypeptides and/or one or any combination of reagents present in a liquid medium. The liquid medium may be an aqueous liquid medium, such as water, a buffered solution, and the like. One or more additives such as a salt (e.g., NaCl, MgCl2, KCI, MgSO4), a buffering agent (a Tris buffer, N-(2-Hydroxyethyl)-piperazine-N′-(2-ethanesulfonic acid) (HEPES), 2-(N-Morpholino)-ethanesulfonic acid (MES), 2-(N-Morpholino)-ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, glycerol, a chelating agent, and the like may be present in such compositions.

The subject compositions may be present in any suitable environment. According to one embodiment, the composition is present in a reaction tube (e.g., a 0.2 mL tube, a 0.6 ml tube, a 1.5 mL tube, or the like) or a well. In certain aspects, the composition is present in two or more (e.g., a plurality of) reaction tubes or wells (e.g., a plate, such as a 6-, 12-, 24-, 48-, 96-or 384-well plate). The tubes and/or plates may be made of any suitable material, e.g., polypropylene, or the like. In certain aspects, the tubes and/or plates in which the composition is present provide for efficient heat transfer to the composition (e.g., when placed in a heat block, water bath, thermocycler, and/or the like), so that the temperature of the composition may be altered within a short period of time, e.g., as necessary for a particular degradation or enzymatic reaction to occur. According to certain embodiments, the composition is present in a thin-walled polypropylene tube, or a plate having thin-walled polypropylene wells.

Other suitable environments for the subject compositions include, e.g., a microfluidic chip (e.g., a “lab-on-a-chip device”). The composition may be present in an instrument configured to bring the composition to a desired temperature, e.g., a temperature-controlled water bath, heat block, or the like. The instrument configured to bring the composition to a desired temperature may be configured to bring the composition to a series of different desired temperatures, each for a suitable period of time (e.g., the instrument may be a thermocycler).

Aspects of the present disclosure also include kits. The kits may include, e.g., one or any combination of reagents for performing the polypeptide sequencing methods of the present disclosure described elsewhere herein. Non-limiting examples of reagents are combinations thereof which may be present in a composition of the present disclosure include UMI-functionalized solid supports, a degradation moiety bearing a reactive group for conjugation to a nucleic acid, a primer comprising a cycle number barcode, Edman degradation reagents (including those for providing the alternative DNA compatible Edman degradation conditions described elsewhere herein), a conjugate comprising a primer conjugated to a binding moiety that specifically binds an amino acid, a conjugate comprising a primer conjugated to a binding moiety that specifically binds an amino acid bearing a post-translational modification, nucleic acid sequencing adapters, and any combination thereof.

According to some embodiments, the subject kits comprise one or any combination of the following reagents: (i) UMI-functionalized solid supports; (ii) a degradation moiety bearing a reactive group for conjugation to a nucleic acid; (iii) a primer comprising a cycle number barcode; (iv) Edman degradation reagents; (v) a conjugate comprising a primer conjugated to a binding moiety that specifically binds an amino acid; (vi) a conjugate comprising a primer conjugated to a binding moiety that specifically binds an amino acid bearing a post-translational modification; and (vii) nucleic acid sequencing adapters. According to some embodiments, the degradation moiety comprises a PITC bearing a reactive group for conjugation to the primer comprising the cycle number barcode. In some instances, the reactive group is a click-chemistry reactive group. In certain embodiments, the Edman degradation reagents comprise BF₃ etherate and an aprotic solvent. According to some embodiments, the Edman degradation reagents comprise triethylamine acetate and N,N-dimethylformamide (DMF). In certain embodiments, one or more of the nucleic acid-based reagents comprise non-natural nucleotides (e.g., 7-deazapurine nucleotides) that stabilize the nucleic acids under Edman degradation conditions. In some instances, the binding moiety is a polypeptide (e.g., an antibody). In other instances, the binding moiety is a small molecule or an aptamer.

Components of the kits may be present in separate containers, or multiple components may be present in a single container. For example, two or more components of the kits may be provided in a single tube, or may be provided in different tubes.

In addition to the above-mentioned components, a kit of the present disclosure may further comprise instructions for using the one or any combination of reagents, e.g., to perform any of the polypeptide sequencing methods of the present disclosure. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, Hard Disk Drive (HDD) etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

For purposes of completeness, the present disclosure is further defined in the following numbered clauses.
1. A method of sequencing a polypeptide, the method comprising:
   (a) labeling the N-terminal amino acid of a polypeptide with a nucleic acid label comprising a unique molecular identifier (UMI);
   (b) degrading the N-terminal amino acid from the polypeptide;
   (c) annealing a primer to the nucleic acid label of the degraded N-terminal amino acid, wherein the primer comprises a barcode corresponding to the identity of the degraded N-terminal amino acid;
   (d) extending the primer annealed to the nucleic acid label to produce an extension product comprising the UMI and the barcode corresponding to the identity of the degraded N-terminal amino acid;
   (e) performing steps (a)-(d) in successive cycles to produce a plurality of extension products, each of the plurality of extension products comprising the UMI, a respective cycle number barcode, and a barcode corresponding to the identity of a respective degraded N-terminal amino acid;
   (f) sequencing the plurality of extension products; and
   (g) determining a sequence of the polypeptide based on the sequences of the plurality of extension products.
2. The method of clause 1, comprising indexing the extension product produced at step (d) with the cycle number barcode.
3. The method of clause 1, wherein step (a) comprises labeling the N-terminal amino acid of the polypeptide with a nucleic acid label comprising the UMI and the cycle number barcode.
4. The method according to any one of clauses 1-3, wherein prior to step (a), the polypeptide is immobilized on a solid support via a nucleic acid attached to the C-terminus of the polypeptide and the surface of the solid support, wherein the nucleic acid comprises the UMI and a primer binding site 3′ or 5′ to the UMI.
5. The method according to clause 4, wherein the labeling step (a) comprises:
   (i) conjugating a degradation moiety to the N-terminal amino acid;
   (ii) conjugating a primer to the degradation moiety, wherein the primer conjugated to the degradation moiety comprises a sequence which is complementary to the primer binding site of the nucleic acid immobilizing the polypeptide to the solid support;
   (iii) annealing the primer conjugated to the degradation moiety to the primer binding site; and
   (iv) extending the primer conjugated to the degradation moiety using the nucleic acid immobilizing the polypeptide to the solid support as the template, thereby labeling the N-terminal amino acid with the nucleic acid label comprising the UMI.
6. The method according to clause 4, wherein the labeling step (a) comprises:
   (i) conjugating to the N-terminal amino acid a degradation moiety conjugated to a primer comprising a sequence which is complementary to the primer binding site of the nucleic acid immobilizing the polypeptide to the solid support;
   (ii) annealing the primer conjugated to the degradation moiety to the primer binding site; and
   (iii) extending the primer conjugated to the degradation moiety using the nucleic acid immobilizing the polypeptide to the solid support as the template, thereby labeling the N-terminal amino acid with the nucleic acid label comprising the UMI.
7. The method according to clause 5 or clause 6, wherein the degradation moiety comprises a phenylisothiocyanate (PITC) bearing a reactive group for conjugation to the nucleic acid comprising the cycle number barcode.
8. The method according to clause 7, wherein the reactive group is a click-chemistry reactive group.
9. The method according to any one of clauses 1-8, wherein the degrading step (b) is performed under conditions comprising a Lewis acid in an aprotic solvent.
10. The method according to clause 9, wherein the Lewis acid is B₃ etherate, BCl₃, BBr₃, Scandium(III) triflate, or any combination thereof.
11. The method according to clause 9, wherein the Lewis acid is BF₃ etherate.
12. The method according to any one of clauses 9-11, wherein the aprotic solvent is acetonitrile, N,N-dimethylformamide (DMF), dimethyl sulfoxide (DMSO), or any combination thereof.
13. The method according to clause 12, wherein the degrading step (b) is performed under conditions comprising triethylamine acetate in N,N-dimethylformamide (DMF).
14. The method according to any one of clauses 1 to 13, wherein one or more nucleic acids employed in step (a) and/or step (b) comprise non-natural nucleotides that stabilize the nucleic acids during degrading step (b).
15. The method according to clause 14, wherein the non-natural nucleotides comprise 7-deazapurine nucleotides.
16. The method according to any one of clauses 1 to 15, wherein at step (c), the primer comprising the barcode corresponding to the identity of the degraded N-terminal amino acid is conjugated to a binding moiety that specifically binds the degraded N-terminal amino acid, and wherein the annealing is dependent upon binding of the binding moiety to the degraded N-terminal amino acid.
17. The method according to clause 16, wherein the binding moiety specifically binds a degraded N-terminal amino acid comprising a post-translational modification, and wherein the barcode indicates the identity of the degraded N-terminal amino acid and the post-translational modification.
18. The method according to clause 16 or clause 17, wherein the post-translational modification is phosphorylation, glycosylation, ubiquitination, nitrosylation, methylation, acetylation, or lipidation.
19. The method according to any one of clauses 16 to 18, wherein the binding moiety is a polypeptide.
20. The method according to clause 19, wherein the polypeptide is an antibody.
21. The method according to any one of clauses 16 to 18, wherein the binding moiety is a small molecule or an aptamer.
22. The method according to any one of clauses 1 to 21, wherein the polypeptide to be sequenced is present in a protein sample isolated from a single cell.
23. The method according to clause 22, wherein the method is a single-cell protein sequencing method performed on a plurality of polypeptides present in the protein sample.
24. The method according to any one of clauses 1 to 23, wherein the polypeptide to be sequenced is present in a protein sample isolated from a tissue sample.
25. The method according to clause 24, wherein the tissue sample is a biopsy sample.
26. The method according to clause 25, wherein the biopsy sample is a tumor biopsy sample.
27. The method according to any one of clauses 1 to 23, wherein the polypeptide to be sequenced is present in a protein sample isolated from a biological fluid.
28. A composition comprising one or any combination of the following:
   (a) UMI-functionalized solid supports,
   (b) a degradation moiety bearing a reactive group for conjugation to a nucleic acid,
   (c) a primer comprising a cycle number barcode,
   (d) Edman degradation reagents,
   (e) a conjugate comprising a primer conjugated to a binding moiety that specifically binds an amino acid,
   (f) a conjugate comprising a primer conjugated to a binding moiety that specifically binds an amino acid bearing a post-translational modification, and
   (g) nucleic acid sequencing adapters.
29. A kit comprising:
   (a) one or any combination of the following reagents:
      (i) UMI-functionalized solid supports,
      (ii) a degradation moiety bearing a reactive group for conjugation to a nucleic acid,
      (iii) a primer comprising a cycle number barcode,
      (iv) Edman degradation reagents,
      (v) a conjugate comprising a primer conjugated to a binding moiety that specifically binds an amino acid,
      (vi) a conjugate comprising a primer conjugated to a binding moiety that specifically binds an amino acid bearing a post-translational modification, and
      (vii) nucleic acid sequencing adapters; and
   (b) instructions for using the one or any combination of reagents to perform the method of any one of clauses 1 to 27.
30. The kit of clause 29, wherein the degradation moiety comprises a PITC bearing a reactive group for conjugation to the primer comprising the cycle number barcode.
31. The kit of clause 30, wherein the reactive group is a click-chemistry reactive group.
32. The kit of any one of clauses 29 to 31, wherein the Edman degradation reagents comprise a Lewis acid in an aprotic solvent.
33. The kit of clause 32, wherein the Lewis acid is BF₃ etherate, BCl₃, BBr3, Scandium(III) triflate, or any combination thereof.
34. The kit of clause 32, wherein the Lewis acid is BF₃ etherate.
35. The kit of any one of clauses 32 to 34, wherein the aprotic solvent is acetonitrile, N,N-dimethylformamide (DMF), dimethyl sulfoxide (DMSO), or any combination thereof.
36. The kit of any one of clauses 29 to 35, wherein one or more of the nucleic acid-based reagents comprise non-natural nucleotides that stabilize the nucleic acids under Edman degradation conditions.
37. The kit of clause 36, wherein the non-natural nucleotides comprise 7-deazapurine nucleotides.
38. The kit of any one of clauses 29 to 37, wherein the binding moiety is a polypeptide.
39. The kit of clause 38, wherein the polypeptide is an antibody.
40. The kit of any one of clauses 29 to 37, wherein the binding moiety is a small molecule or an aptamer.

The following examples are offered by way of illustration and not by way of limitation.

### EXPERIMENTAL

### Example 1—Development of an Alternative Edman Degradation Reaction Compatible With DNA

Traditional Edman degradation conditions are not compatible with DNA. Cleavage and conversion reactions are carried out by neat trifluoroacetic acid (TFA) and TFA solution at elevated temperature, respectively. The use of a strong protic acid poses the greatest challenge to DNA, which is susceptible to depurination under acidic conditions. Observed here was that the cleavage reaction condition degraded a poly(dT) oligonucleotide. Furthermore, PITC and its derivatives that are used to modify the N-terminus of peptides may react with exocyclic amine of nucleobases.

Described in this example is the development of an alternative Edman degradation reaction compatible with DNA. It was hypothesized that the degradation of DNA is mainly caused by protonation of nucleobases under strong acidic conditions. Therefore, an Edman degradation procedure that uses BF₃ etherate in aprotic solvent for the cleavage step was adopted. Consistent with the hypothesis, polypyrimidine sequences were stable under these conditions for extensive periods of time. However, native purine nucleotides still underwent depurination under this condition, albeit at a significantly slower rate. To further enhance the stability of the DNA, the chemically modified purine nucleotides were investigated. 7-Deazapurine nucleotides lack the nitrogen atom at the 7-position and are reported to be resistant to depurination.

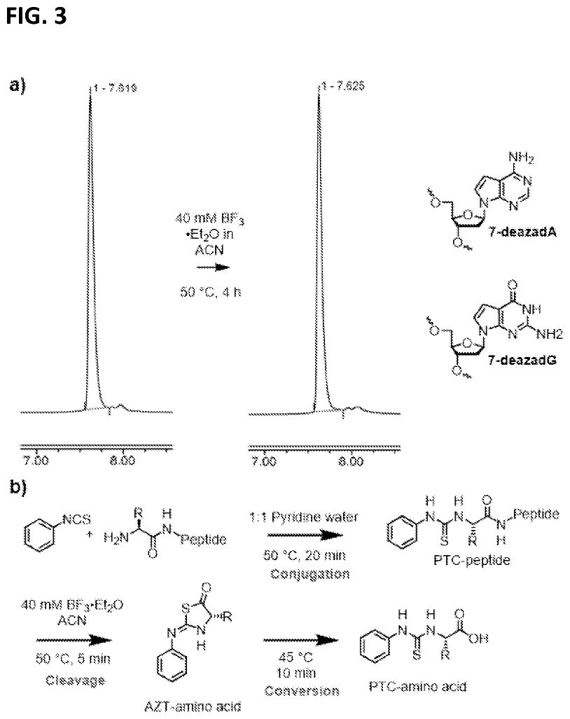

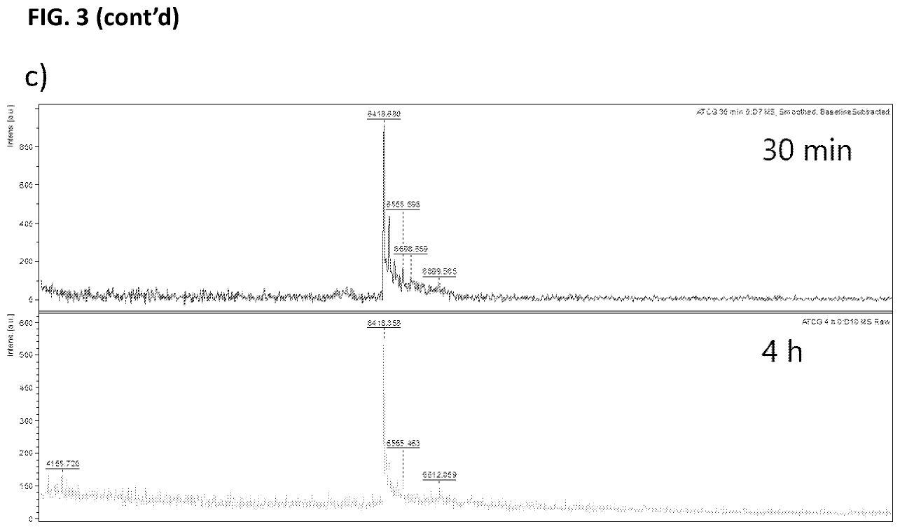

Oligonucleotides containing 7-deazapurine nucleotides were stable under the degradation conditions for a duration of 4 h, which is validated by LC and MS (FIGS. 3A and 3C). Given the rapid N-terminus amino acid cleavage in the presence of BF₃ etherate (vide infra), the stability of 7-deazapurine modified DNA is sufficient for the Edman degradation process. Finally, 7-deazapurine modified DNA was subjected to PITC in water/pyridine (1:1) at 50° C. for 16 h, and no modification of DNA was detected. This is consistent with the low nucleophilicity of exocyclic amine of nucleobases.

Cleavage of N-terminus amino acids went to completion in 5 min in the presence of 40 mM BF₃ etherate. The cleavage induced by BF₃ etherate is significantly faster than the TFA cleavage reaction, which takes 30 min. Anilinothiazolinone (ATZ) amino acids are the major fragments generated under this condition, and ATZ amino acids were converted to stable PTC amino acids under mild basic conditions (FIG. 3B). PTC amino acids can be readily synthesized by reacting PITC or its derivatives with amino acids, and thus allow easy access to target molecules for binding agent (e.g., antibody) generation.

### Example 2-Development of Conditions for Solid Phase Edman Degradation of Dna-Peptide Conjugates

This example relates to turning the DNA-compatible Edman degradation to a solid phase format. Solid phase reactions bring two benefits. Firstly, solid phase reactions allow the use of a large excess of reagents which can be easily removed by filtration. This greatly simplifies the design of the iterative Edman degradation cycle. Further, DNA conjugated peptides have different reactivities in organic solvents compared to the nonconjugated peptides due to the insolubility of oligonucleotides in these solvents, which has been well documented in the field of DNA encoded libraries. Initial experiments performed here are consistent with the literature and suggest that Edman degradation does not occur on DNA conjugated PTC-peptide in anhydrous acetonitrile. It has been reported that immobilization of DNA on a solid phase makes chemical transformations in nonaqueous solvent become accessible to DNA-encoded synthesis. In view of this, Edman degradation of peptide-DNA conjugates on solid supports was investigated.

As demonstrated herein, Edman degradation can be performed on an immobilized DNA-peptide conjugate. To test Edman degradation on solid supports, it was first established that the stability of DNA under degradation was not affected by immobilization. Next, DNA-peptide conjugates were synthesized on solid supports to test the feasibility of Edman degradation. This was achieved by synthesizing dibenzocyclooctyne (DBCO) modified DNA sequence on controlled pore glass (CPG), or polystyrene coated carboxylic magnetic beads. CPGs are commonly used for solid-phase DNA synthesis, and magnetic beads are generally compatible with enzymes and allow easy separation. A PTC-peptide containing a C-terminal azidolysine was conjugated with DBCO modified DNA via a strain-promoted alkyne-azide cycloaddition (SPAAC) to form a model DNA-peptide conjugate (FIG. 4A). Cleavage reactions were carried out with 40 mM BF₃ etherate in anhydrous acetonitrile. The supernatants were collected, and the release of PTC amino acid was confirmed by LC-MS on both solid supports (FIG. 4B). DNA-peptide conjugates were cleaved from CPG post degradation and analyzed by HPLC. The results suggested that the degradation went to completion in 10 min (FIG. 4C).

### Example 3—Barcoding of Degradation Fragments of the N-terminus Amino Acid on a Model Peptide

In this example, the DNA compatible Edman degradation reaction described above will be utilized to achieve the first cycle of INDEED. To achieve this, 7-deazapurine modified DNA (UMI) with a 3′-amino and a 5′-DBCO group will first be synthesized. The UMI is immobilized on 1 μm Carboxylic Acid Dynabeads™ by carbodiimide chemistry. A polypeptide containing a C-terminal azidolysine is conjugated to the DNA by SPAAC. Second, the N-terminus of the polypeptide is modified with a PITC derivative (2) bearing an alkyne. Third, methyltetrazine azide (3) is conjugated with the alkyne by copper(I)-catalyzed azide alkyne cycloaddition (CuAAC), and a trans-cyclooctene (TCO) modified primer is installed via inverse electron demand Diels-Alder (IEDDA) reaction. In the fourth step, the DNA UMI is transcribed by primer extension reaction. In the final step, the N-terminus amino acid is cleaved from the peptide by treating with BF₃ etherate (FIG. 5B). This step is achieved by a cleavage-hydrolysis tandem reaction which generates a PTC amino acid fragment barcoded with DNA UMI.

Experiments performed to investigate several key steps for DNA barcoding of degradation fragments determined that Edman degradation can be performed on the complete UMI-peptide-primer construct. Peptide-UMI conjugate was immobilized on beads using the conditions established above. Alkyne modified PITC (2) was synthesized by first treating 4-(2-aminoethyl)aniline with alkyne NHS ester (1) at 0 ° C. to achieve selective acylation of the aliphatic amine. The aniline moiety was subsequently converted to isothiocyanate under the conditions reported by Scattolin et al. (FIG. 5A). Modification of peptide-UMI conjugate on beads by (2) was quantitative. Primer was successfully installed by the aforementioned CuAAC-IEDDA cascade to yield the complete UMI-peptide-primer construct (FIG. 5B). The relative amount of primer sequences on beads can be quantified by flow cytometry via annealing a fluorescently labeled complementary strand. The yield of Edman degradation can be determined by comparing the fluorescent intensity before and after the reaction. The degradation yield was approximately 85% after 10 min (FIG. 5C).

Next, it was determined that 7-Deazapurine modified DNA is accepted by DNA polymerases. Barcoding of PTC amino acids requires the polymerase to accept 7-deazapurine nucleotide substituted template-primer duplexes and 7-deazapurine nucleoside triphosphates as substrates. We examined primer extension by Sequenase version 2.0, Klenow (exo-), and Bst 3.0 (FIG. 5D). All these polymerases were able to incorporate 7-deazapurine nucleotide triphosphates efficiently, and Sequenase version 2.0 will be used in future experiments.

### Example 4—Alternative Method of Barcoding of Degradation Fragments of the N-terminus Amino Acid on a model peptide

In this example, a 3′-dibenzocyclooctyne (DBCO) modified deazapurine substituted DNA template was immobilized onto magnetic beads (FIG. 5F). Subsequently, a 7-amino acid long model peptide with a C-terminal azidolysine was conjugated to the DNA via a strain-promoted alkyne-azide cycloaddition (SPAAC). Azide modified PITC (4, FIG. 5E) reacted with the N-terminus of the model peptide.

To introduce a primer for barcode transfer, the conjugation of an alkyne-modified primer to the azide group on 4 was employed. SPAAC was opted for due to the susceptibility of the phenylthiocarbamyl group in 2 to oxidation, which may lead to undesired reactions with reactive oxygen species generated during copper(I)-catalyzed azide-alkyne cycloaddition (CuAAC). One drawback of SPAAC is its relatively slow reaction rate. However, it was determined that the reaction rate could be enhanced by hybridizing the template and the primer, which increased the effective concentration of reactants. For instance, the conjugation of the primer in the presence of a 7-amino acid long peptide was remarkably completed within 10 minutes.

After incorporating the primer, it was extended using Klenow (FIG. 5F), and the beads were subjected to Lewis acid catalyzed Edman degradation. Taken advantage of during this step was the insolubility of DNA in acetonitrile, which allows DNA-barcoded ATZ amino acids remain hybridized with the template DNA following the initial cleavage reaction. Subsequently, a basic conversion reaction serves two purposes: (i) it converts ATZ amino acids into stable PTC amino acids, and (ii) it denatures the duplex DNA, releasing the DNA-barcoded PTC amino acids. Additionally, DTT was introduced to inhibit the oxidative degradation of PTC amino acids under basic conditions. This cleavage and conversion reaction yielded an overall yield of 96%, and the resulting structure was confirmed through mass spectrometry analysis (FIG. 5G).

### Example 5—Characterization of Binding Agents That Recognize Ptc Amino Acids

BD-PEX serves the function of converting the binding events between DNA barcoded PTC amino acids and binding agents (e.g., antibodies) to DNA output. PTC amino acids retain the structural features of the original amino acids and only differ by the PTC modification on the amino group. Because these amino groups are often modified to conjugate with carrier proteins during generation of antibodies against amino acids, it is expected that antibodies raised against amino acids will also recognize PTC amino acids. In turn, it is expected that commercially available antibodies may be used to detect these amino acids. A fingerprint of four amino acids is sufficient for identification of most proteins within the human proteome.

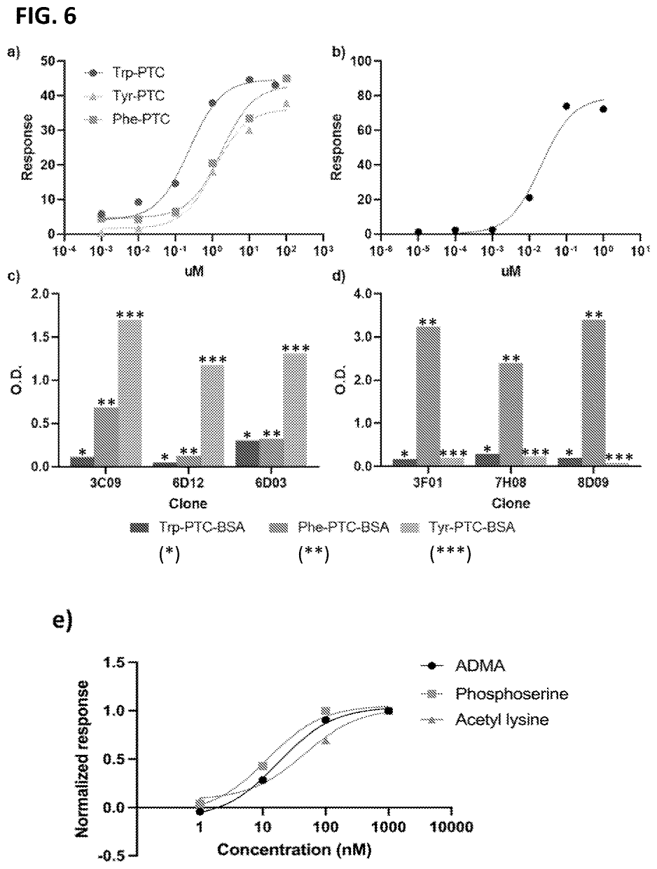

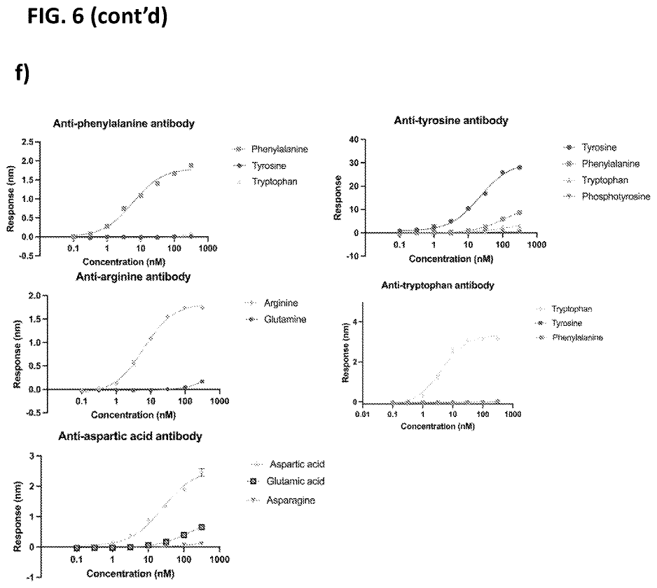

Demonstrated first in this example is that antibodies against amino acids recognize PTC amino acids. As proof of principle, PTC-tryptophan was synthesized by reacting PITC derivative (1) and tryptophan and conjugated to azide modified DNA. Other than the difference in the linker that is distal to PTC-tryptophan, this conjugate is structurally identical to that formed by INDEED. The binding affinity of a commercially available tryptophan mAb to DNA conjugated PTC-tryptophan was measured by biolayer interferometry (BLI) and surface plasmon resonance (SPR). The tryptophan mAb has a Kd of 280 nM against PTC-tryptophan and the binding is highly specific, as no binding was observed with PTC-tyrosine and PTC-phenylalanine (FIG. 6A). Further, an antibody against phosphotyrosine (PY20) was also tested and a Kd of 20 nM was obtained (FIG. 6B). This indicates that PTC amino acids bearing post-translational modifications (PTMs) may also be recognized by their corresponding anti-PTM antibodies. Additional anti-PTM antibodies were tested, resulting in the identification of antibodies that recognize PTC for asymmetric dimethylarginine (ADMA), acetyl lysine, and phosphoserine (FIG. 6E).

As demonstrated above, PTC amino acids bearing a click handle can be readily synthesized and conjugated to azide modified carrier proteins, such as BSA. Demonstrated next in this example is that antibodies against PTC amino acids can be generated. To obtain new PTC amino acid specific antibodies, an antibody discovery campaign was initiated. PTC-tyrosine and PTC-phenylalanine were synthesized and conjugated to azide modified BSA. Mice were challenged with modified BSA, and strong immune responses were observed for both antigens. Subsequently, antibody-producing B cells were harvested and fused to form hybridomas. In the ensuing subcloning and screening, several candidates with high specificity were identified (FIGS. 6C and 6D). Using the strategy, antibodies against PTC modified phenylalanine, tyrosine, tryptophan, arginine and aspartic acid were identified (FIG. 6F)

### Example 6—Development of Binding Dependent Primer Extension (BD-PEX) to Convert dna Barcoded Degradation Fragments to dna Sequences

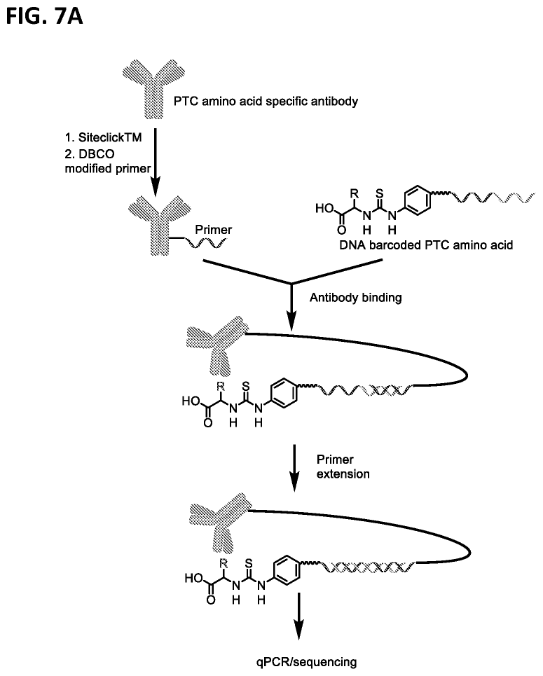

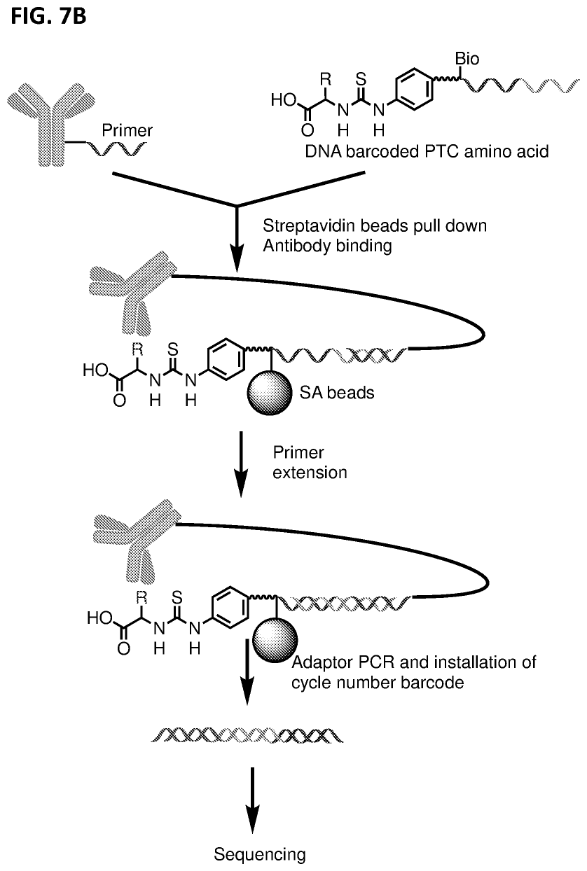

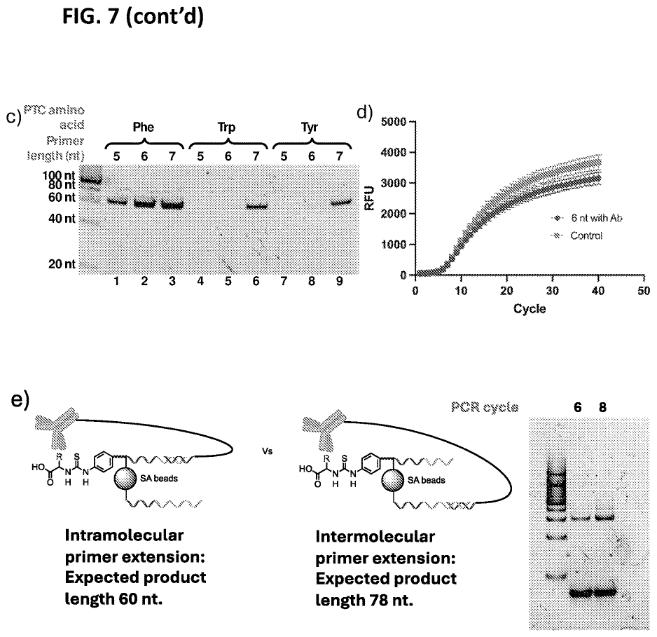

Highly specific binding agents (e.g., antibodies) will be used to recognize PTC amino acids released by DNA encoded Edman degradation, and the binding event will be converted to DNA output. Conversion to DNA output can reveal all antibody-antigen interactions simultaneously, and the resulting DNA can be further amplified to increase the detection sensitivity. To achieve this, and with reference to FIG. 7A, the Fc region of the antibodies will be site-specifically modified using the SiteClick™ kit to introduce azide functionalities. Subsequently, a DBCO modified primer bearing an antibody specific barcode is conjugated to the antibody. The primer sequence is designed to be short, and thus disfavors intermolecular primer extension. The binding of antibody to its PTC amino acid target will increase the effective molarity of the primer facilitating the duplex formation between the complementary sequences on the primer and the template. The resulting complex can serve as the substrate for primer extension which converts the binding event to sequenceable DNA output. The primer extension product may be analyzed by qPCR and/or DNA sequencing to determine reaction yield and the limit of detection.

### Example 7—Binding Dependent Primer Extension (bd-pex) to Convert dna Barcoded Degradation Fragments to dna Sequences Using Biotinylated Primers

Described in this example is the use of biotinylated primers during the INDEED process, enabling the barcoded DNA to be pulled down by streptavidin beads, and BD-PEX can be carried out on the beads (FIG. 7B). To achieve this, and with reference to FIG. 7B, the Fc region of the antibodies were site-specifically modified using kits such as SiteClick™ kit, or oYo-Link kit to introduce a click handle, e.g., azide or tetrazine. Subsequently, a DBCO or TCO modified primer bearing an antibody specific barcode was conjugated to the antibody. The stability of the primer-template complex plays a crucial role in governing the efficiency and specificity of intramolecular primer extension. While increasing the length of the primer can improve the efficiency of primer extension, longer primers also tend to promote extension in the absence of an antibody-antigen recognition event. It was discovered that primers with a length of 6 nucleotides allowed for efficient primer extension and simultaneously minimizing non-specific primer extension (FIG. 7C). Furthermore, intermolecular primer extension served as another source of non-specific primer extension on the beads. This form of non-specific primer extension could be effectively inhibited by reducing the surface density of DNA-barcoded PTC amino acids. At a density of 1 pmol of DNA per mg of magnetic beads, intermolecular primer extension was virtually eliminated (FIG. 7E).

### Example 8—Sequencing/Fingerprinting of Short Peptides

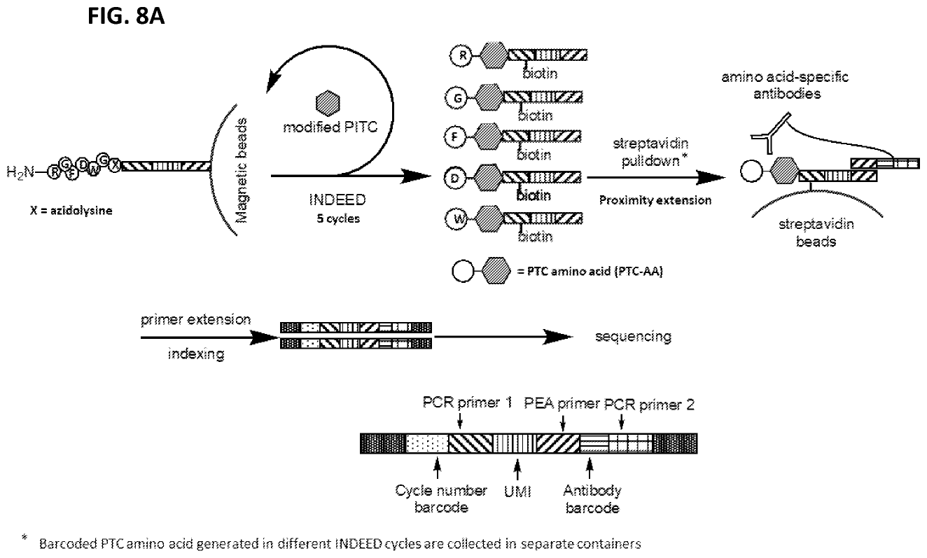

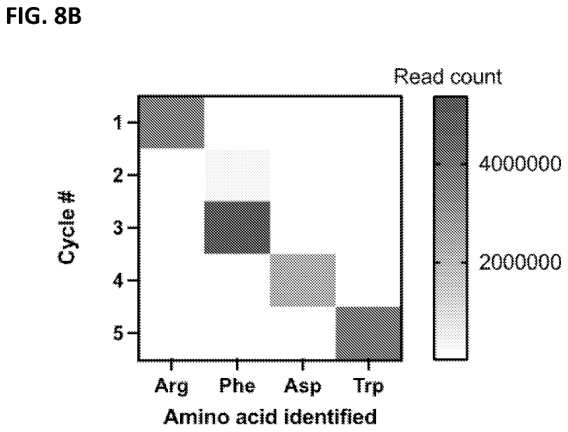

A peptide having the sequence RGFDWGK{N₃} was subjected to five cycles of the INDEED process. The resulting DNA barcoded PTC amino acids from each cycle were pulled down onto streptavidin beads in separate containers. Proximity primer extension was carried out with a mixture of DNA barcoded PTC amino acid specific antibodies (100 nM of each of anti PTC-Arg antibody, anti PTC-Phe antibody, anti PTC-Asp antibody, anti PTC-Trp antibody). After primer extension, adaptor PCR was performed in each vessel with adaptor primers bearing cycle number barcode. Finally, all the DNA was pooled, indexed and sequenced on a MiSeq sequencer (FIG. 8A). The cycle number barcodes and antibody barcodes were extracted from the sequencing results. The read counts of all possible combinations of barcodes were plotted on a heatmap (FIG. 8B), and the result was consistent with sequence of the peptide.

### Example 9—Sequencing/Fingerprinting of Short Peptides and Quantification of Single Amino Acid Substitutions

Single amino acid substitutions are caused by non-synonymous single nucleotide polymorphisms (nsSNPs) and often disrupt function of proteins by altering protein structure. DNA sequencing allows sensitive detection of SNPs, but detection of single amino acid substitutions by MS is often limited by sensitivity. It is expected that the methods described herein will enable detection of single amino acid substitutions by sequencing peptides at single amino acid resolution. Furthermore, DNA sequencing readout will allow signal amplification and thus enhance the sensitivity.

To demonstrate the peptide sequencing via INDEED, a mixture of peptides that bear a single amino acid substitution will be immobilized on UMI coated magnetic beads using the chemistry established above. INDEED will be carried out iteratively, and DNA barcoded PTC amino acids will be collected. Organic solvent from the degradation fragment mixture will be removed by solid phase extraction or buffer exchange before the enzymatic reaction. Combined DNA barcoded PTC amino acids are then converted to DNA sequences by BD-PEX using a mixture of DNA barcoded antibodies against PTC amino acids. The resulting DNA will be sequenced. It is expected that single amino substitutions can be identified regardless of their position within the polypeptides. In addition to identifying single amino acid substitutions in a highly parallel fashion, the present sequencing method will allow quantification of these variations using read count. If each polypeptide molecule is uniquely barcoded, the occurrence of single amino acid substitutions can be quantified digitally, and the quantification will not be affected by PCR bias.

### Example 10—Mapping and Quantification of Post-Translational Modifications (PTMs) in Model Peptides

Identification and quantification of PTMs are crucial for understanding protein function. Currently, PTMs are commonly studied using antibody-based techniques and mass spectrometry. However, antibody-based techniques are often not site-specific. While PTM analysis by MS can provide information on the site of modification, accurate quantitation of PTMs often requires the use of chemically synthesized isotopically labeled peptide standards. It is expected that the majority of PTM specific antibodies may be adopted in the sequencing methods of the present disclosure. Because the PTC amino acid fragments are barcoded with DNA containing the information regarding the order of amino acids within the peptide, the present method can map PTM sites specifically. Furthermore, quantification of PTMs can be achieved by DNA sequencing without the need for synthesizing isotopically labeled peptide standards specific to the protein of interest.

To demonstrate this capability, a peptide containing two tyrosine amino acids and all of its possible phosphotyrosine derivatives will be synthesized. INDEED will be performed on the mixture of these peptides. Tyrosine will be identified by an antibody that is specific to PTC-tyrosine, and phosphotyrosine will be recognized by anti-phosphotyrosine antibody, such as PY20 demonstrated above. The recognition events will be recorded by BD-PEX, and the resulting DNA will be sequenced. It is expected that the site of phosphorylation will be encoded in the DNA sequence and the relative abundance of phosphorylation will be quantifiable using the read count. The method may be expanded to other PTMs such as phosphorylation on serine and threonine, methylation, acylation, and glycosylation depending on the stability of PTMs during INDEED.

### Example 11—Fingerprinting of Full-length Proteins

Proteins in eukaryotes are, on average, 400 amino acids long. Due to limitations in degradation efficiency, fingerprinting full-length proteins by Edman degradation may not be preferable. Thus, in order to fingerprint full-length proteins, the protein may be digested by endopeptidases, such as trypsin, to yield short peptides that are then subjected to INDEED. This capability will be demonstrated by fingerprinting the trypsin digest of a full-length protein.

Immobilization of peptides via cysteine may be employed thanks to the wide range of cysteine-specific reactions such as a-halocarbonyls and maleimides. By controlling pH, selective modification of cysteine over other nucleophilic residues such as lysine, histidine, and N-terminus can be achieved. However, the low abundance of cysteine (2%) may lead to incomplete capture of tryptic peptides. The C-terminal carboxylic acid is a more generalizable conjugation handle for peptide immobilization. It has been reported that C-terminal carboxylic acid can be selective labeled by carboxypeptidase, the proteolysis activity of which is inhibited at high pH while the transpeptidation activity catalyzes the ligation of a nucleophilic molecule to the C-terminal carboxylic acid. More recently, photoredox-catalyzed decarboxylation of C-terminal carboxylic acids has been described. Although this method has only been demonstrated for short peptides that are less than 10 amino acids long, it may serve as a more general and efficient method for C-terminus immobilization. Conjugation of click handles such as alkynes has been demonstrated, and thus these methods can be readily implemented to the INDEED workflow.

The side chains of cysteine and lysine may be capped prior to degradation. It is well documented that cysteine can be capped by alkylation. Capping of lysine may be achieved by first masking N-terminus with a reversible modification, and lysines are subsequently irreversibly capped by reagents such as NHS ester. After capping of lysine, N-terminal amino groups are released by removal of the reversible modification. Furthermore, these capping reactions can be used to introduce affinity tags that are recognized by existing affinity reagents, and thus further expand the scope of sequenceable amino acids.

### Example 12—Mapping of Proteoforms From Single Cells

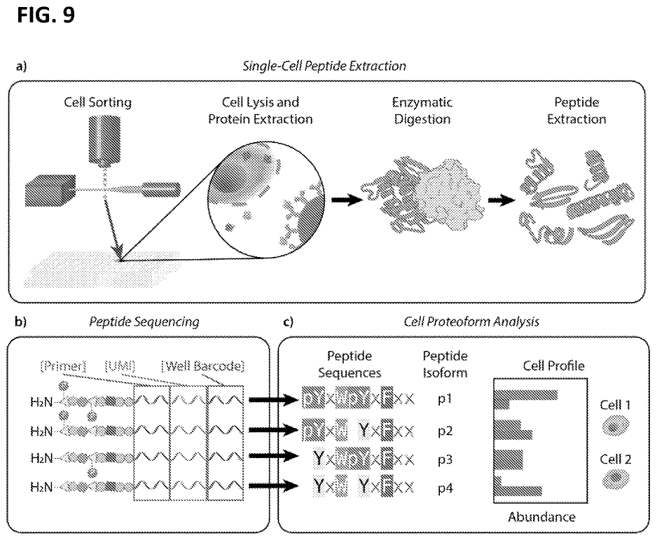

Mapping proteoforms at the single-cell level may reveal cell heterogeneity beyond the gene or even protein level, and may greatly advance our understanding of cell functions, organism development, and disease mechanisms. The polypeptide sequencing methods of the present disclosure may be used for single-molecule profiling of proteoforms such as single amino acid substitutions, and post-translational modifications. Here, a workflow for mapping these proteoforms at the single-cell level will be developed (FIG. 9). First, to isolate and enrich proteins of interest, single cells are isolated via FACS in multi-well plates containing lysis buffer, and beads coated with antibodies against proteins of interest. Second, the proteins of interest are eluted from antibody coated beads and digested by trypsin. Finally, the resulting peptides are conjugated to DNA UMI and sequenced using our single-molecule peptide sequencing technique. Importantly, UMIs used in this workflow may also include barcodes that are specific to each well, and thus allow identification and quantification of proteoforms in each cell.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of sequencing a polypeptide, the method comprising:
(a) labeling the N-terminal amino acid of a polypeptide with a nucleic acid label comprising a unique molecular identifier (UMI);
(b) degrading the N-terminal amino acid from the polypeptide;
(c) annealing a primer to the nucleic acid label of the degraded N-terminal amino acid, wherein the primer comprises a barcode corresponding to the identity of the degraded N-terminal amino acid;
(d) extending the primer annealed to the nucleic acid label to produce an extension product comprising the UMI and the barcode corresponding to the identity of the degraded N-terminal amino acid;
(e) performing steps (a)-(d) in successive cycles to produce a plurality of extension products, each of the plurality of extension products comprising the UMI, a respective cycle number barcode, and a barcode corresponding to the identity of a respective degraded N-terminal amino acid;
(f) sequencing the plurality of extension products; and
(g) determining a sequence of the polypeptide based on the sequences of the plurality of extension products.

2. The method of claim 1, comprising indexing the extension product produced at step (d) with the cycle number barcode.

3. The method of claim 1, wherein step (a) comprises labeling the N-terminal amino acid of the polypeptide with a nucleic acid label comprising the UMI and the cycle number barcode.

4. The method according to any one of claims 1-3, wherein prior to step (a), the polypeptide is immobilized on a solid support via a nucleic acid attached to the C-terminus of the polypeptide and the surface of the solid support, wherein the nucleic acid comprises the UMI and a primer binding site 3′ or 5′ to the UMI.

5. The method according to claim 4, wherein the labeling step (a) comprises:
(i) conjugating a degradation moiety to the N-terminal amino acid;
(ii) conjugating a primer to the degradation moiety, wherein the primer conjugated to the degradation moiety comprises a sequence which is complementary to the primer binding site of the nucleic acid immobilizing the polypeptide to the solid support;
(iii) annealing the primer conjugated to the degradation moiety to the primer binding site; and
(iv) extending the primer conjugated to the degradation moiety using the nucleic acid immobilizing the polypeptide to the solid support as the template, thereby labeling the N-terminal amino acid with the nucleic acid label comprising the UMI.

6. The method according to claim 4, wherein the labeling step (a) comprises:
(i) conjugating to the N-terminal amino acid a degradation moiety conjugated to a primer comprising a sequence which is complementary to the primer binding site of the nucleic acid immobilizing the polypeptide to the solid support;
(ii) annealing the primer conjugated to the degradation moiety to the primer binding site; and
(iii) extending the primer conjugated to the degradation moiety using the nucleic acid immobilizing the polypeptide to the solid support as the template, thereby labeling the N-terminal amino acid with the nucleic acid label comprising the UMI.

7. The method according to claim 5 or claim 6, wherein the degradation moiety comprises a phenylisothiocyanate (PITC) bearing a reactive group for conjugation to the nucleic acid comprising the cycle number barcode.

8. The method according to claim 7, wherein the reactive group is a click-chemistry reactive group.

9. The method according to any one of claims 1-8, wherein the degrading step (b) is performed under conditions comprising a Lewis acid in an aprotic solvent.

10. The method according to claim 9, wherein the Lewis acid is BF₃ etherate, BCl₃, BBr₃, Scandium(III) triflate, or any combination thereof.

11. The method according to claim 9, wherein the Lewis acid is BF₃ etherate.

12. The method according to any one of claims 9-11, wherein the aprotic solvent is acetonitrile, N,N-dimethylformamide (DMF), dimethyl sulfoxide (DMSO), or any combination thereof.

13. The method according to claim 12, wherein the degrading step (b) is performed under conditions comprising triethylamine acetate in N,N-dimethylformamide (DMF).

14. The method according to any one of claims 1 to 13, wherein one or more nucleic acids employed in step (a) and/or step (b) comprise non-natural nucleotides that stabilize the nucleic acids during degrading step (b).

15. The method according to claim 14, wherein the non-natural nucleotides comprise 7-deazapurine nucleotides.

16. The method according to any one of claims 1 to 15, wherein at step (c), the primer comprising the barcode corresponding to the identity of the degraded N-terminal amino acid is conjugated to a binding moiety that specifically binds the degraded N-terminal amino acid, and wherein the annealing is dependent upon binding of the binding moiety to the degraded N-terminal amino acid.

17. The method according to claim 16, wherein the binding moiety specifically binds a degraded N-terminal amino acid comprising a post-translational modification, and wherein the barcode indicates the identity of the degraded N-terminal amino acid and the post-translational modification.

18. The method according to claim 16 or claim 17, wherein the post-translational modification is phosphorylation, glycosylation, ubiquitination, nitrosylation, methylation, acetylation, or lipidation.

19. The method according to any one of claims 16 to 18, wherein the binding moiety is a polypeptide.

20. The method according to claim 19, wherein the polypeptide is an antibody.

21. The method according to any one of claims 16 to 18, wherein the binding moiety is a small molecule or an aptamer.

22. The method according to any one of claims 1 to 21, wherein the polypeptide to be sequenced is present in a protein sample isolated from a single cell.

23. The method according to claim 22, wherein the method is a single-cell protein sequencing method performed on a plurality of polypeptides present in the protein sample.

24. The method according to any one of claims 1 to 23, wherein the polypeptide to be sequenced is present in a protein sample isolated from a tissue sample.

25. The method according to claim 24, wherein the tissue sample is a biopsy sample.

26. The method according to claim 25, wherein the biopsy sample is a tumor biopsy sample.

27. The method according to any one of claims 1 to 23, wherein the polypeptide to be sequenced is present in a protein sample isolated from a biological fluid.

28. A composition comprising one or any combination of the following:
(a) UMI-functionalized solid supports,
(b) a degradation moiety bearing a reactive group for conjugation to a nucleic acid,
(c) a primer comprising a cycle number barcode,
(d) Edman degradation reagents,
(e) a conjugate comprising a primer conjugated to a binding moiety that specifically binds an amino acid,
(f) a conjugate comprising a primer conjugated to a binding moiety that specifically binds an amino acid bearing a post-translational modification, and
(g) nucleic acid sequencing adapters

29. A kit comprising:
(a) one or any combination of the following reagents:
(i) UMI-functionalized solid supports,
(ii) a degradation moiety bearing a reactive group for conjugation to a nucleic acid,
(iii) a primer comprising a cycle number barcode,
(iv) Edman degradation reagents,
(v) a conjugate comprising a primer conjugated to a binding moiety that specifically binds an amino acid,
(vi) a conjugate comprising a primer conjugated to a binding moiety that specifically binds an amino acid bearing a post-translational modification, and
(vii) nucleic acid sequencing adapters; and
(b) instructions for using the one or any combination of reagents to perform the method of any one of claims 1 to 27.

30. The kit of claim 29, wherein the degradation moiety comprises a PITC bearing a reactive group for conjugation to the primer comprising the cycle number barcode.

31. The kit of claim 30, wherein the reactive group is a click-chemistry reactive group.

32. The kit of any one of claims 29 to 31, wherein the Edman degradation reagents comprise a Lewis acid in an aprotic solvent.

33. The kit of claim 32, wherein the Lewis acid is BF₃ etherate, BCl₃, BBr₃, Scandium(III) triflate, or any combination thereof.

34. The kit of claim 32, wherein the Lewis acid is BF₃ etherate.

35. The kit of any one of claims 32 to 34, wherein the aprotic solvent is acetonitrile, N,N-dimethylformamide (DMF), dimethyl sulfoxide (DMSO), or any combination thereof.

36. The kit of any one of claims 29 to 35, wherein one or more of the nucleic acid-based reagents comprise non-natural nucleotides that stabilize the nucleic acids under Edman degradation conditions.

37. The kit of claim 36, wherein the non-natural nucleotides comprise 7-deazapurine nucleotides.

38. The kit of any one of claims 29 to 37, wherein the binding moiety is a polypeptide.

39. The kit of claim 38, wherein the polypeptide is an antibody.

40. The kit of any one of claims 29 to 37, wherein the binding moiety is a small molecule or an aptamer.
